(19) United States
(12) Patent Application Publication
(10) Pub. No.: US 2010/0003291 A1
(43) Pub. Date: Jan. 7, 2010

(54) NANO-PARTICLES FOR COSMETIC APPLICATIONS

(75) Inventor: Ginger Denison Rothrock, Durham, NC (US)

Correspondence Address: MORGAN, LEWIS & BOCKIUS LLP, 1701 MARKET STREET, PHILADELPHIA, PA, 19103-2921, US

(73) Assignee: Liquidia Technologies, Inc., Research Triangle Park, NC (US)

(21) Appl. No.: 12/300,964

(22) PCT Filed: May 15, 2007
(86) PCT No.: PCT/US2007/011752

§ 371 Date: May 14, 2009

Related U.S. Application Data
(60) Provisional application No. 60/800,478, filed on May 15, 2006.

(51) Int. Cl. A61K 8/02

(52) U.S. Cl. 424/401; 977/926; 977/773

(57) ABSTRACT

Micro and/or nano-particles are fabricated in micro and/or nano-scale cavities of replicate molds for cosmetic applications. The micro and/or nano-particles can be fabricated for inclusion in cosmetic composition or fabricated from cosmetic ingredients.

### CROSS REFERENCE TO RELATED APPLICATION(S)

This application is based on and claims priority to U.S. Provisional application 60/800,478, filed May 15, 2006, which is incorporated herein by reference in its entirety.

### INCORPORATION BY REFERENCE

All documents referenced herein are hereby incorporated by reference as if set forth in their entirety herein, as well as all references cited therein, including the document(s) in Appendix A.

### TECHNICAL FIELD

Generally, this invention relates to micro and/or nano scale particles and methods for fabrication same for cosmetic applications. More specifically, the particles have substantially uniform size and shape and are fabricated for cosmetic applications or from cosmetic ingredients.

### BACKGROUND

It is estimated that there are approximately 100,000 personal care products on the market in 2006. Of these 100,000 personal care products roughly 10,000 products include nano-scale ingredients such as micronized particles, fullerenes, quantum dots, liposomes and other commercially available in nano sized chemicals.

The cosmetics industry uses nano-scale ingredients routinely. Some reasons that nano-scale ingredients are becoming more and more popular in the cosmetic industry is that due to their small size and extremely high ratio of surface area to volume, nanotechnology materials often have chemical or physical properties that may different from those of their larger counterparts including increased chemical and biological activity. Some typical products relying on nano-sized components include sunscreen, make-up, hair care products, lotions, gels, and the like.

Currently, however, the nano-scale ingredients used by the cosmetic industry has drawbacks in that the precise size, uniformity of the size, shape, and uniformity of the shape are not controllable parameters beyond naturally occurring nano-scale systems such as liposomes. Therefore, it would be beneficial to provide a system and ingredients that can offer nano-scale ingredients of virtually any shape and have high uniformity among the shapes of a given sample.

### SUMMARY

An object of the present invention is to provide micro and/or nano-particles of predetermined three dimensional geometric shape for cosmetic applications and methods for making such particles. In some embodiments, a cosmetic composition includes a dispersion of particles in a cosmetically acceptable medium where substantially every particle of the particles is configured and dimensioned into a predetermined three dimensional geometric shape and has a broadest cross-sectional dimension of less than about 100 micrometers. In some embodiments, the particles are fabricated from a composition of cosmetic ingredients. According to some embodiments, the particle is substantially a cube, a column, a cylinder, a cone, a sphere, or the like.

In alternative embodiments, the particle is less than about 75 micrometers in the broadest dimension, less than about 50 micrometers in the broadest dimension, less than about 25 micrometers in the broadest dimension, less than about 10 micrometers in the broadest dimension, less than about 5 micrometers in the broadest dimension, less than about 1 micrometer in the broadest dimension, less than about 750 nm in the broadest dimension, less than about 500 nm in the broadest dimension, less than about 250 nm in the broadest dimension, less than about 200 nm in the broadest dimension, less than about 150 nm in the broadest dimension, less than about 100 nm in the broadest dimension, less than about 75 nm in the broadest dimension, less than about 50 nm in the broadest dimension, or less than about 25 nm in the broadest dimension.

According to some embodiments, a cosmetic composition of the present invention includes a cosmetic film having a film layer and a plurality of structures associated with the film layer wherein substantially every structure of the structures is configured and dimensioned into a predetermined three dimensional geometric shape and has a broadest cross-sectional dimension of less than about 100 micrometers. In some embodiments, the film layer and the structures are formed from the same cosmetic ingredients. In alternative embodiments, the film layer is formed from a different composition than that of the structures.

The present invention also includes methods for making the cosmetic particles of the present invention. In some embodiments, a method for forming cosmetic particles includes the steps of providing a replica mold defining cavities having substantially uniform three dimensional geometric shapes, introducing a cosmetic substance into the cavities of the replica mold, hardening the substance in the cavities of the replica mold such that a particle of the cosmetic substance is formed in the cavity, and removing the particle from the cavity of the replica mold. In some embodiments, the replica mold includes a low surface-energy polymeric material. In other embodiments, the replica mold includes a fluoropolymer.

### BRIEF DESCRIPTION OF THE DRAWINGS

Reference is made to the accompanying drawings in which are shown illustrative embodiments of the presently disclosed subject matter, from which its novel features and advantages will be apparent.

FIGS. 1A-1H show fabrication of cosmetic particles and cosmetic film according to embodiments of the present invention;

FIG. 2 shows 200 nm trapezoidal particles made from various matrix materials according to an embodiment of the present invention;

FIG. 3 shows fabrication of PEG particles of different shapes according to an embodiment of the present invention;

FIG. 4 shows a DLS trace with the value measured for particle size as 0.62±0.2 μm, the line indicates monodispersity of the particles with no aggregation occurring according to an embodiment of the present invention;

FIG. 5 shows 200 nm harvested PEG particles according to an embodiment of the present invention;

FIG. 6 shows harvested particles on film by dragging a blade across the surface to yield rolled up film according to an embodiment of the present invention;

FIG. 7 shows particles embedded in an adhesive layer according to an embodiment of the present invention;

FIG. 8 shows Bosch-type etch lines on particles according to an embodiment of the present invention;

FIG. 9 shows harvested 2×2×1 μm positively charged particles containing fluorescent oligonucleotide condensed inside according to an embodiment of the present invention;

FIG. 10 shows the image of FIG. 9 imaged by both DIC (left) and fluorescent light microscopy (right) according to an embodiment of the present invention;

FIG. 11 shows SEM images of oligonucleotides in positively charged particles according to an embodiment of the present invention;

FIG. 12 shows a fluorescent microscopy image of harvested 2×2×1 micrometer neutral particles containing a fluorescent oligonucleotide inside according to an embodiment of the present invention;

FIG. 13 shows DIC (left) and Fluorescent light microscopy (right) images of harvested 2×2×1 μm neutral PEG-based particles containing a fluorescent oligonucleotide inside according to an embodiment of the present invention;

FIG. 14 shows particles observed after separation of a mold and treated silicon wafer using scanning electron microscopy (SEM) and optical microscopy according to an embodiment of the present invention;

FIG. 15 shows a schematic of CDI-Activated particles with a PEG matrix for ligand attachment according to an embodiment of the present invention;

FIG. 16 shows SEM images of patterned TiO2 xerogel according to an embodiment of the present invention; and

FIG. 17 shows SEM images of patterned TiO2 (anatase form) after calcination at 450° C. according to an embodiment of the present invention.

### DETAILED DESCRIPTION

One aspect of the present invention provides particles of and/or for cosmetic compositions that are fabricated in substantially uniform three dimensional geometric shapes and of substantially uniform sizes. In another aspect of the present invention, cosmetic films are fabricated that include a surface or surfaces with micro and/or nano-scale three dimensional geometric structures. The cosmetic particles of the present invention are molded into micro and/or nano-scale structures by using precision micro and/or nano-scale replicate molds fabricated from a patterned master. The patterned master, which can be in some embodiments an etched silicon wafer, includes predetermined micro and/or nano-scale structures that become replicated in the micro and/or nano-scale replicate molds and in which the cosmetic particles are formed.

In some embodiments, the micro and/or nano-scale structures of the patterned master can be any three dimensional geometric shape that can be fabricated into a master. The micro and/or nano-scale structures can be arranged into arrays that can include a plurality of repetitive structures or a variety of different three dimensional geometric shapes. The arrays can also be organized symmetrically, in a staggered pattern, offset, or some combination thereof. In some embodiments, the arrays of micro and/or nano-scale three dimensional geometric structures can also have a variety of features, sizes, shapes, compositions, or the like assorted within each array.

The present subject matter will now be described more fully hereinafter with reference to the accompanying Figures and Examples, in which representative embodiments are shown. It will be appreciated that the present disclosure can be embodied in different forms and should not be construed as limited to the embodiments set forth herein. Rather, these embodiments are provided so that this disclosure will be thorough and complete, and will fully convey the scope of the embodiments to those skilled in the art. Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety. Throughout the specification and claims, a given chemical formula or name shall encompass all optical and stereoisomers, as well as racemic mixtures where such isomers and mixtures exist. All publications, patent applications, and patents referenced herein are hereby incorporated herein by reference in their entirety.

### Formation of Isolated Micro- and/or Nano-Particles for Cosmetic Applications

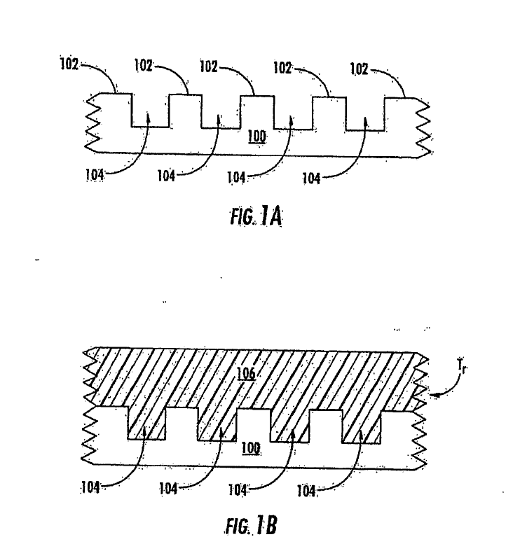

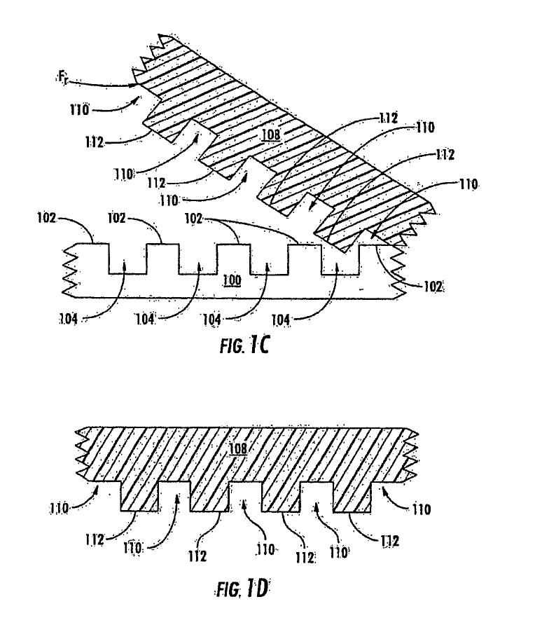

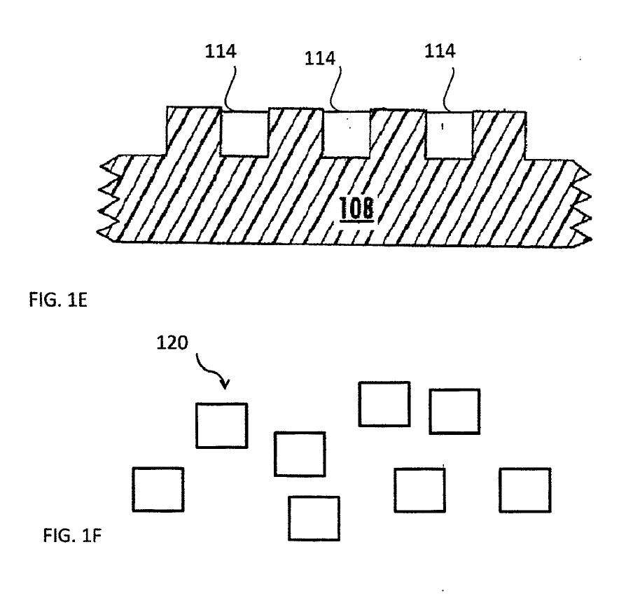

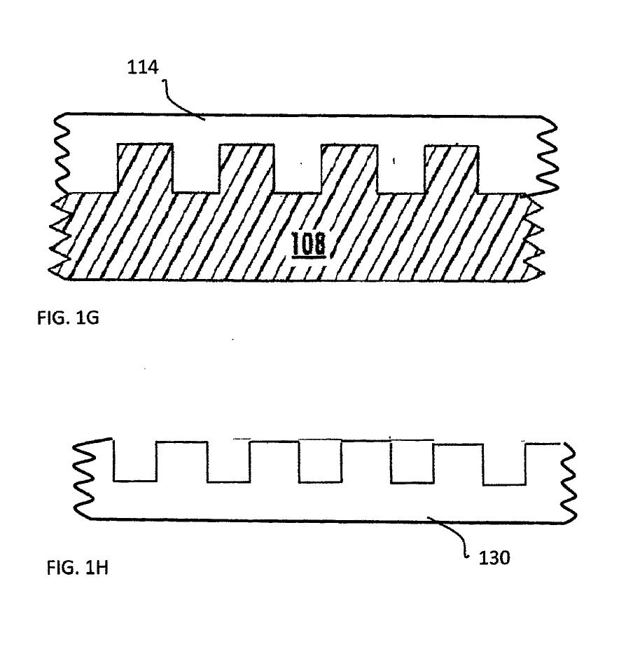

In some embodiments, the present invention provides methods for making isolated three dimensional geometric micro and/or nano-particles for inclusion in cosmetic applications or of cosmetic ingredients. In some embodiments, the process of fabricating the micro and/or nano-particles includes initially forming a replicate mold. Referring now to FIGS. 1A-1D, an exemplary embodiment of fabricating a cosmetic nano-scale particle is shown. Initially a patterned master 100 is provided. In some embodiments, patterned master 100 can be an etched substrate, such as a silicon wafer, that is etched in the desired pattern such as a predetermined three dimensional geometric shape to be replicated. Patterned master 100 includes a plurality of non-recessed surface areas 102 and a plurality of recesses 104.

Referring now to FIG. 1B, a liquid material 106, for example, a liquid composition, such as FLUOROCUR™ (Liquidia Technologies, Inc.), is then introduced to patterned master 100. In some embodiments, liquid material 106 can be a liquid or semiliquid material that coats the surface of patterned master 100. After liquid material 106 is introduced to patterned master 100, liquid material 106 is treated by treating process Tᵣ, to cure, solidify, harden, or the like liquid material 106 and form replicate mold 108 of patterned master 100. In some embodiments, treating process Tᵣ cures, hardens, solidifies or the like by, for example, exposure to UV light, actinic radiation, thermal energy, combinations thereof, or the like.

Referring now to FIGS. 1C and 1D, a force Fr is applied to treated liquid material to remove it from patterned master 100. As shown in FIGS. 1C and 1D, replicate mold 108 includes a plurality of cavities 110, which are mirror images of the plurality of non-recessed surface areas 102 of patterned master 100. Replicate mold 108 can now be used for the fabrication of isolated three dimensional geometric micro and/or nano-structures for cosmetic applications, as shown in FIG. 1E.

Next, substance 114 to be formed into the micro and/or nano-particles of the present invention is introduced to replicate mold 108. Substance 114 fills cavities 110 and can be hardened, solidified, cured, or the like in cavities 110 to form particles 120 (FIG. 1F). Because particles 120 are formed in cavities 110 of replicate molds 108 particles 120 assume the three dimensional geometric shape of cavities 110. Substance 114 can include, in some embodiments, cosmetic ingredients such that particles 120 include the cosmetic ingredients of a cosmetic product. In other embodiments, substance 114 can include a composition to be added to a cosmetic composition to adjust a consistency, delivery, application, suspension, combinations thereof, or the like of the cosmetic composition.

In other embodiments, as shown in FIG. 1G, substance 114 can be applied to replicate mold 108 such that excess substance 114 exists between and connecting substance 114 in respective cavities 110. After substance 114 is hardened, solidified, cured, or the like, substance 114 can be removed from replicate mold 108 and used as a cosmetic film 130. Cosmetic film 130 includes a surface having three dimensional geometric structures fabricated thereon.

According to other embodiments, alternative materials and methods for fabricating the isolated micro and/or nano-particles of the present invention can be found in published PCT International Patent Application Serial no.'s PCT/US04/42706 filed Dec. 20, 2004; PCT/US04/31274 filed Sep. 23, 2004; PCT/US05/04421 filed Feb. 14, 2005; PCT/US06/23722 filed Jun. 19, 2006; PCT/US06/31067 filed Aug. 9, 2006; PCT/US06/34997 filed Sep. 7, 2006; PCT/US06/43305 filed Nov. 7, 2006; PCT/US07/002,476 filed Jan. 29, 2007; and the PRINT™ processes (Liquidia Technologies, Inc.), each of which is incorporated herein by reference in their entirety.

According to some embodiments, the material from which replicate mold 108 is formed, such as FLUOROCUR™, has a surface energy below about 30 mN/m. According to another embodiment the surface energy of the replicate mold material is between about 10 mN/m and about 20 mN/m. According to another embodiment, the replicate mold material has a low surface energy of between about 12 mN/m and about 15 mN/m. In some embodiments, the replicate mold material has a surface energy lower than about 18 mN/m. In some embodiments, the replicate mold material has a surface energy lower than about 15 mN/m. According to a further embodiment the replicate mold material has a surface energy less than about 12 mN/m. According to another embodiment, the replicate mold material has a low surface energy less than about 10 mN/m.

According to some embodiments, cavities 110 of replicate molds 108 can be configured to assist or facilitate receipt of the substance to be formed into the three dimensional geometric shape micro and/or nano-particles. Variables such as, for example, the surface energy of the replicate mold materials, the relative difference in surface energies of the replicate mold materials and that of the substance to be molded, the volume of the cavity, the diameter of the opening of the cavity relative to the cavities depth, the permeability of the replicate mold material, the viscosity of the substance to be molded as well as other physical and chemical properties of the substance to be molded can interact and affect the willingness of the recess to receive the substance to be molded.

According to some embodiments, replicate molds 108, including cavities 110, can be fabricated from materials disclosed herein such as, for example, low surface energy polymeric materials. Because the material of the mold has such low surface energy the substance to be molded into particles for cosmetic compositions or from cosmetic ingredients does not wet the surface of the replicate mold. The substance to be molded does, however, fill the cavities of the replicate mold. In other embodiments, the replicate mold can be dipped into the substance to be molded to fill the cavities. In other embodiments, the cavities of the replicate molds can be filled by positioning a droplet of the substance to be molded onto the mold surface and allow the droplet to travel around on the surface of the replicate mold. As the volume of the substance passes over the cavities, subvolumes of the substance enter and fill the cavities.

According to other embodiments, a voltage can assist in introducing the substance to be molded into cavities of a replicate mold. In further embodiments, other factors that can influence the filling of cavities include, but are not limited to, recess volume, diameter, surface area, surface energy, contact angle between a substance to be molded and the material of the recess, voltage applied across a substance to be molded, temperature, environmental conditions surrounding the replicate mold such as for example the removal of oxygen or impurities from the atmosphere, combinations thereof, and the like.

### Three Dimensional Geometric Micro and/or Nano-Particles

According to some embodiments the three dimensional geometric micro and/or nano-particle of the present invention is fabricated with a desired predetermined shape and is less than about 500 μm in a given dimension (e.g. minimum, intermediate, or maximum dimension). The predetermined shape can be determined, in some embodiments, by the shape and/or structure that patterned master 100 is fabricated into. Particle can be of an organic material or an inorganic material and can be one uniform compound or component or a mixture of compounds or components. According to some embodiments, a particle is composed of a matrix that has a predetermined surface energy.

According to some embodiments, particles 120 formed in cavities 110 of replicate mold 108 are less than about 500 μm in a dimension. In other embodiments, the particle is less than about 450 μm in a broadest dimension. In other embodiments, the particle is less than about 400 μm in a broadest dimension. In other embodiments, the particle is less than about 350 μm in a broadest dimension. In other embodiments, the particle is less than about 300 μm in a broadest dimension. In other embodiments, the particle is less than about 250 μm in a broadest dimension. In other embodiments, the particle is less than about 200 μm in a broadest dimension. In other embodiments, the particle is less than about 150 μm in a broadest dimension. In other embodiments, the particle is less than about 100 μm in a broadest dimension. In other embodiments, the particle is less than about 75 μm in a broadest dimension. In other embodiments, the particle is less than about 50 μm in a broadest dimension. In other embodiments, the particle is less than about 25 μm in a broadest dimension. In other embodiments, the particle is less than about 10 μm in a broadest dimension. In other embodiments, the particle is less than about 5 μm in a broadest dimension. In other embodiments, the particle is less than about 1 μm in a broadest dimension.

According to alternative embodiments, the particles 120 formed in cavities 110 of replicate molds are less than about 900 nm in a broadest dimension. According to alternative embodiments, the particles 120 formed in cavities 110 of replicate molds are less than about 800 nm in a broadest dimension.

According to alternative embodiments, the particles 120 formed in cavities 110 of replicate molds are less than about 700 nm in a broadest dimension. According to alternative embodiments, the particles 120 formed in cavities 110 of replicate molds are less than about 600 nm in a broadest dimension.

According to alternative embodiments, the particles 120 formed in cavities 110 of replicate molds are less than about 500 nm in a broadest dimension.

According to alternative embodiments, the particles 120 formed in cavities 110 of replicate molds are less than about 400 nm in a broadest dimension.

According to alternative embodiments, the particles 120 formed in cavities 110 of replicate molds are less than about 300 nm in a broadest dimension.

According to alternative embodiments, the particles 120 formed in cavities 110 of replicate molds are less than about 250 nm in a broadest dimension.

According to alternative embodiments, the particles 120 formed in cavities 110 of replicate molds are less than about 200 nm in a broadest dimension.

According to alternative embodiments, the particles 120 formed in cavities 110 of replicate molds are less than about 150 nm in a broadest dimension.

According to alternative embodiments, the particles 120 formed in cavities 110 of replicate molds are less than about 100 mm in a broadest dimension.

According to alternative embodiments, the particles 120 formed in cavities 110 of replicate molds are less than about 75 nm in a broadest dimension.

According to alternative embodiments, the particles 120 formed in cavities 110 of replicate molds are less than about 50 nm in a broadest dimension. According to alternative embodiments, the particles 120 formed in cavities 110 of replicate molds are less than about 40 nm in a broadest dimension. According to alternative embodiments, the particles 120 formed in cavities 110 of replicate molds are less than about 35 nm in a broadest dimension.

According to alternative embodiments, the particles 120 formed in cavities 110 of replicate molds are less than about 30 nm in a broadest dimension. According to alternative embodiments, the particles 120 formed in cavities 110 of replicate molds are less than about 25 nm in a broadest dimension. According to alternative embodiments, the particles 120 formed in cavities 110 of replicate molds are less than about 20 nm in a broadest dimension.

In yet further embodiments, the particle is less than about 1 μm in dimension. According to some embodiments the particle is between about 1 nm and about 500 nm in a dimension. According to other embodiments, the particle is between about 10 nm and about 200 nm in a dimension. In still further embodiments, the particle is between about 80 nm and 120 nm in a dimension. According to still more embodiments the particle is between about 20 nm and about 120 nm in dimension. The dimension of the particle can be a predetermined dimension, a cross-sectional diameter, a circumferential dimension, or the like.

A plurality of cosmetic particles 120 of the present invention can, in some embodiments, have substantially uniform three dimensional geometric shape and/or size. The plurality of particles 120 can include substantially the same predetermined geometric shape or a variety of predetermined three dimensional geometric shapes. In alternate embodiments, the plurality of particles 120 includes polydispersity in broadest dimension of less than about 1.0010, less than about 1.0008, less than about 1.0006, or less than about 1.0005.

According to some embodiments, particles 120 of many predetermined regular and irregular shape and size configurations can be made with the materials and methods of the presently disclosed subject matter. Examples of representative particle shapes that can be made using the materials and methods of the presently disclosed subject matter include, but are not limited to, non-spherical, spherical, viral shaped, bacteria shaped, cell shaped, rod shaped (e.g., where the rod is less than about 200 nm in diameter), chiral shaped, right triangle shaped, flat shaped (e.g., with a thickness of about 2 nm, disc shaped with a thickness of greater than about 2 nm, or the like), boomerang shaped, combinations thereof, and the like.

In some embodiments, the composition of the particles 120 is predetermined, such as the location and orientation of chemical components within the individual isolated three dimensional geometric micro and/or nano particle. Such particles 120 can be fabricated by methods of the present invention to modify or control performance of the isolated three dimensional geometric micro and/or nano-particle by rationally structuring the isolated three dimensional geometric micro and/or nano-particle so that it is optimized for a particular application. In some embodiments, the method includes incorporating biological targeting agents into the micro and/or nano-particles for tissue, protein, hair, skin, cell, or the like augmentation, enhancement, alteration, restoration, treatment or the like. In some embodiments, the method includes designing the particles to include a specific biological recognition motif. In some embodiments, the biological recognition motif includes biotin/avidin and/or other proteins.

In some embodiments, the method includes tailoring the chemical composition of the micro and/or nano-particle to fabricate a particle for a cosmetic composition. In some embodiments, the particles are designed and synthesized so that recognition elements are located on the surface of the particle where the core of the particle is preserved for sustained release or activation under certain controlled or desired conditions.

In some embodiments, the material from which the particles are formed includes, without limitation, compositions or components useful in cosmetic products such as, for example, sunscreens, hair care products, make-up products, mascara, and the like. In other embodiments, the material from which the particles are formed include, but are not limited to, one or more of a polymer, a liquid polymer, a solution, a monomer, a plurality of monomers, a polymerization initiator, a polymerization catalyst, an inorganic precursor, an organic material, a natural product, a metal precursor, a pharmaceutical agent, a magnetic material, a paramagnetic material, a ligand, a cell penetrating peptide, a porogen, a surfactant, a plurality of immiscible liquids, a solvent, a charged species, combinations thereof, or the like.

In some embodiments, the monomer includes butadienes, styrenes, propene, acrylates, methacrylates, vinyl ketones, vinyl esters, vinyl acetates, vinyl chlorides, vinyl fluorides, vinyl ethers, acrylonitrile, methacrylnitrile, acrylamide, methacrylamide allyl acetates, fumarates, maleates, ethylenes, propylenes, tetrafluoroethylene, ethers, isobutylene, fumaronitrile, vinyl alcohols, acrylic acids, amides, carbohydrates, esters, urethanes, siloxanes, formaldehyde, phenol, urea, melamine, isoprene, isocyanates, epoxides, bisphenol A, alcohols, chlorosilanes, dihalides, dienes, alkyl olefins, ketones, aldehydes, vinylidene chloride, anhydrides, saccharide, acetylenes, naphthalenes, pyridines, lactams, lactones, acetals, thiiranes, episulfide, peptides, derivatives thereof, and combinations thereof.

In yet other embodiments, the polymer includes polyamides, proteins, polyesters, polystyrene, polyethers, polyketones, polysulfones, polyurethanes, polysiloxanes, polysilanes, cellulose, amylose, polyacetals, polyethylene, glycols, poly(acrylate)s, poly(methacrylate)s, poly(vinyl alcohol), poly(vinylidene chloride), poly(vinyl acetate), poly(ethylene glycol), polystyrene, polyisoprene, polyisobutylenes, poly(vinyl chloride), poly(propylene), poly(lactic acid), polyisocyanates, polycarbonates, alkyds, phenolics, epoxy resins, polysulfides, polyimides, liquid crystal polymers, heterocyclic polymers, polypeptides, conducting polymers including polyacetylene, polyquinoline, polyaniline, polypyrrole, polythiophene, and poly(p-phenylene), dendimers, fluoropolymers, derivatives thereof, combinations thereof.

In still further embodiments, the material from which the particles are formed includes a non-wetting agent. According to another embodiment, the material is a liquid material in a single phase. In other embodiments, the liquid material includes a plurality of phases. In some embodiments, the liquid material includes, without limitation, one or more of multiple liquids, multiple immiscible liquids, surfactants, dispersions, emulsions, micro-emulsions, micelles, particulates, colloids, porogens, active ingredients, combinations thereof, or the like.

In some embodiments, additional components are included with the material of the particle to functionalize the particle. According to some embodiments, the modification components of the particle can be encased within the particle, partially encased within the particle, on the exterior surface of the particle, combinations thereof, or the like. Additional components can include, but are not limited to, drugs, biologics, more than one drug, more than one biologic, combinations thereof, and the like.

In some embodiments, the particle includes a biodegradable polymer. In other embodiments, the particle composition is modified to be a biodegradable polymer (e.g., a poly(ethylene glycol) that is functionalized with a disulfide group). In some embodiments, the biodegradable polymer includes, without limitation, one or more of a polyester, a polyanhydride, a polyamide, a phosphorous-based polymer, a poly(cyanoacrylate), a polyurethane, a polyorthoester, a polydihydropyran, a polyacetal, combinations thereof, or the like.

In some embodiments, the polyester includes, without limitation, one or more of polylactic acid, polyglycolic acid, poly(hydroxybutyrate), poly(ε-caprolactone), poly(β-malic acid), poly(dioxanones), combinations thereof, or the like. In some embodiments, the polyanhydride includes, without limitation, one or more of poly(sebacic acid), poly(adipic acid), poly(terpthalic acid), combinations thereof, or the like. In yet other embodiments, the polyamide includes, without limitation, one or more of poly(imino carbonates), polyaminoacids, combinations thereof, or the like.

According to some embodiments, the phosphorous-based polymer includes, without limitation, one or more of a polyphosphate, a polyphosphonate, a polyphosphazene, combinations thereof, or the like. Further, in some embodiments, the biodegradable polymer further includes a polymer that is responsive to a stimulus. In some embodiments, the stimulus includes, without limitation, one or more of pH, radiation, ionic strength, oxidation, reduction, temperature, an alternating magnetic field, an alternating electric field, combinations thereof, or the like. In some embodiments, the stimulus includes an alternating magnetic field.

In some embodiments, the present subject matter provides functionalized micro and/or nano-particles and methods for functionalizing isolated micro- and/or nano-particles. In one embodiment, the functionalization includes introducing chemical functional groups to a surface either physically or chemically. In some embodiments, the method of functionalization includes introducing at least one chemical functional group to at least a portion of microparticles and/or nanoparticles.

### Harvesting the Three Dimensional Geometric Particles

In some embodiments, the three dimensional geometric particle must be removed or harvested from cavity 110 after it is fabricated therein. The particle can be harvested by a number of approaches, including but not limited to applying a surface that has an affinity for the particle that is greater than an affinity between the particle and cavity 110 of replicate mold 108. In other embodiments, replicate mold 108 can be deformed such that the particle is released from cavity 110. In other embodiments, replicate mold 108 can be swelled with a first solvent to extrude the particle. In other embodiments, the replicate mold 108 can be washed with a second solvent that has an affinity for the particles or that puts the particles into solution, thereby removing the particles from cavities 110.

In some embodiments, other mechanisms used to harvest the particles from cavities 110 include mechanical force, ultrasonic forces, megasonic forces, electrostatic forces, or magnetic force means. In some embodiments, the harvesting or collecting of the particles includes a process selected from the group including scraping with a doctor blade, a brushing process, a dissolution process, an ultrasound process, a megasonics process, an electrostatic process, and a magnetic process. In some embodiments, the harvesting or collecting of the particles includes applying a material to at least a portion of a surface of the particle wherein the material has an affinity for the particles. In some embodiments, the material includes an adhesive or sticky surface. In some embodiments, the material includes, without limitation, one or more of a carbohydrate, an epoxy, a wax, polyvinyl alcohol, polyvinyl pyrrolidone, polybutyl acrylate, a polycyano acrylate, a polyacrylic acid and polymethyl methacrylate. In some embodiments, the harvesting or collecting of the particles includes cooling water to form ice (e.g., in contact with the particles).

In some embodiments, the plurality of particles includes a plurality of monodisperse particles. In some embodiments, the particle or plurality of particles is selected from the group including a semiconductor device, a crystal, a drug delivery vector, a gene delivery vector, a disease detecting device, a disease locating device, a photovoltaic device, a porogen, a cosmetic, an electret, an additive, a catalyst, a sensor, a detoxifying agent, an abrasive, such as a CMP, a micro-electro-mechanical system (MEMS), a cellular scaffold, a taggant, a pharmaceutical agent, and a biomarker. In some embodiments, the particle or plurality of particles include a freestanding structure.

In some embodiments, the surface that has an affinity for the particles includes an adhesive or sticky surface (e.g. carbohydrates, epoxies, waxes, polyvinyl alcohol, polyvinyl pyrrolidone, polybutyl acrylate, polycyano acrylates, polymethyl methacrylate). In some embodiments, the liquid is water that is cooled to form ice. In some embodiments, the water is cooled to a temperature below the Tm of water but above the Tg of the particle. In some embodiments the water is cooled to a temperature below the Tg of the particles but above the Tg of the mold or substrate. In some embodiments, the water is cooled to a temperature below the Tg of the mold or substrate.

In some embodiments, the first solvent includes supercritical fluid carbon dioxide. In some embodiments, the first solvent includes water. In some embodiments, the first solvent includes an aqueous solution including water and a detergent. In embodiments, the deforming the surface element is performed by applying a mechanical force to the surface element. In some embodiments, the method of removing the patterned structure further includes a sonication method.

According to yet another embodiment the particles are harvested on a fast dissolving substrate, sheet, or films. The film can further include water, plasticizing agents, natural and/or artificial flavoring agents, sulfur precipitating agents, saliva stimulating agents, cooling agents, surfactants, stabilizing agents, emulsifying agents, thickening agents, binding agents, coloring agents, sweeteners, fragrances, combinations thereof, and the like.

According to some embodiments, a method for harvesting particles from a replicate mold includes the use of a sacrificial layer that has an affinity for particles. Additional methods and materials for harvesting can be found in the published patent applications incorporated herein by reference.

### Particles for Cosmetic Applications

According to some embodiments, particles of the present invention are fabricated as a component of a cosmetic composition. In other embodiments, particles of the present invention make up all or substantially all of a cosmetic composition. In some embodiments, examples of cosmetic compositions include, pressed powder, foundation, blush, eye-shadow, cosmetic sticks, mascara, reflective or iridescent particles for application to the skin of an individual, combinations thereof, and the like. In some embodiments the particles include, pigments, such as melanin. In some embodiments, the cosmetic application enhances beauty, masks skin blemishes, alters a natural coloring, or the like.

In further embodiments, the particles of the present invention are formulated to impart water resistance properties to a cosmetic composition. In an embodiment, a cosmetic composition that includes the particles of the present invention resists running, smudging, or the like when interfaced with water. In other embodiments, the particles impart long-lasting properties or anti-transfer properties for cosmetic compositions.

In some embodiments, particles contain melamine-formaldehyde or urea-formaldehyde for making up the skin, softening defects of the relief of the skin (wrinkles or pores), treating greasy skin, and the like.

In some embodiments, particles are fabricated as hollow thermoplastic particles composed of one of more of the following: acrylonitrile, acrylics, styrenes, vinylidene chloride, combinations thereof, and the like for cosmetic applications.

In some embodiments, the particles contain an indoline-based product, silicone components, wax, cubic gel, ascorbic acid, sebum-absorbing compounds, inorganic fillers, polyorganosiloxane containing at least one oxyethylenated group, polyamide, anti-irritant properties, a keratin (hair, eyebrows, eyelashes, nails) setting or styling composition, and the like for cosmetic applications.

In some embodiments, particles according to the present invention include particles made of a film-forming polymer where the particles can form a film by themselves or in the presence of at least one plasticizer, e.g., materials that soften synthetic polymers. Plasticizers useful in the practice of the invention include lecithin, polysorbates, dimethicone copolyol, glycols, citrate esters, glycerin, dimethicone, and other similar ingredients disclosed in the International Cosmetic Ingredient Dictionary and Handbook Vol. 4 (9^{th} ed. 2002), more particularly the plasticizers disclosed on page 2927, which is hereby incorporated by reference.

In some embodiments, the particles are non-film forming polymer. In some embodiments, particles form a percolation network (as this term is used in U.S. Pat. No. 6,126,929, issued Oct. 3, 2000 and incorporated herein by reference in the entirety) in the matrix of a film. In some embodiments, nonfilm-forming polymers are polymers such as “JONCRYL® SCX 8082”, “JONCRYL® 90” by the company JOHNSON POLYMER; “NEOCRYL® XK 52” by the company AVECIA RESINS; and “RHODOPAS® 5051” by the company RHODIA CHIMIE. In some embodiments, aqueous dispersions of film-forming adherent polymer include acrylic dispersions sold under the names NEOCRYL XK-90®, NEOCRYL A-1070®, NEOCRYL A-1090®, NEOCRYL BT-62®, NEOCRYL A-1079®, NEOCRYL A-523® by the company AVECIA-NEORESINS, DOW LATEX 432® by the company DOW CHEMICAL, DAITOSOL 5000 AD® by the company DAITO KASEY KOGYO; or else the aqueous dispersions of polyurethane which are sold under the names NEOREZ R-981®, NEOREZ R-974® by the company AVECIA-NEORESINS, AVALURE UR-405®, AVALURE UR-410®, AVALURE UR-425®, AVALURE UR-450®, SANCURE 875®, SANCURE 861®, SANCURE 878®, SANCURE 2060® by the company GOODRICH, IMPRANIL 85® by the company BAYER, AQUAMERE H-1511® by the company HYDROMER. Exemplary dispersions of film-forming polymer in the liquid fatty phase, in the presence of stabilizing agents, are described in the documents EP-A-749746, EP-A-923928, and EP-A-930060, the disclosures of which are specifically incorporated by reference herein.

In some embodiments, particles according to methods of the present invention include particles for treating or coating keratin fibers such as eyebrows, eyelashes, hair, combinations thereof, and the like. According to some embodiments, particles include, but are not limited to, a lipophilic organofluorine compound, hair styling compositions including adhesive particles for holding or shaping hair, bleaching ingredients for hair, reshapeable hair-styling compositions containing (meth)acrylic copolymer particles, nail polish containing anionic particles particularly of polyester and/or polyurethane, mineral containing particles, polyalkyleneimine containing particles.

In some embodiments, particles according to the present invention include lotion particles, soap particles, deodorant particles, shaving particles, dermatology particles. According to some embodiments, the particles are organic particles containing at least one cationic polymer, such as a cationic polymer containing amine groupings. In some embodiments, particles containing a cationic polymer are dispersed readily. In some embodiments, particles containing a cationic polymer are dispersed readily and evenly in fatty binders. In some embodiments, compositions that include particles of the present invention provide good adhesion to skin and/or good cohesion properties when compacted. In some embodiments, the particles contain at least one amphoteric polymer, such that these compositions are easily dispersable, have good stability, and/or good adhesion to skin. In some embodiments, the particles include polyamide particles dispersed in a skin-cleansing composition. In some embodiments, the particles include deformable hollow particles, such as particles made from an acrylic or styrene based monomer, acrylonitrile, vinylidene chloride, combinations thereof, and the like. In some embodiments, the particles are fabricated from a water-absorbing ingredient, wherein combinations of such particles may be made from hydrophilic and lipophilic compositions. In some embodiments, particles of the present invention are fabricated from or contain an organopolysiloxane, a cubic gel, ascorbic acid, one or more sebum-absorbing compounds, at least one inorganic filler, combinations thereof, or the like.

In some embodiments, particles of the present invention are included in an emulsion. Particles included in an emulsion include face wash, lotions, liquid makeup, shampoos, and other hair styling products, polyamide particles, hollow thermoplastic particles composed of, for example, acrylonitrile, acrylics, styrenes, and vinylidene chloride, anti-wrinkle compositions, sunscreen particles such as metal oxides, i.e., TiO2 and combinations thereof. In some embodiments, particles for UV protection applications can include particles in a transparent composition that reflects infrared radiation, particles containing Bismuth oxychloride, particles containing Zirconium, particles containing ceramics, combinations thereof, and the like.

In one embodiment, the composition may contain sunscreens. Sunscreens may be inorganic nanoparticles or organic compounds. In one embodiment the nanoparticles are inorganic compounds composed essentially of metal oxides. Suitable metal oxides comprise one or more of iron oxide, aluminum oxide, zirconium oxide, vanadium oxide, niobium oxide, tantalum oxide, chromium oxide, molybdenum oxide, tungsten oxide, cobalt oxide, nickel oxide, cerium cupric oxide, zinc oxide, tin oxide, antimony oxide titanium dioxide and mixtures thereof, among others. In yet another embodiment titanium dioxide and zinc oxide are used. Without being limited to theory, in most cases the metal oxide nanoparticles provide a sun protection benefit by diffracting the ultraviolet light. The elemental size of 1 nanoparticle is typically from less than 1 m in size, including from about 100 nm to about 500 nm, including about 200 nm to about 350 nm.

Sunscreens according to this invention which are chemical absorbers actually absorb harmful ultraviolet radiation. It is well known that chemical absorbers are classified, depending on the type of radiation they protect against, as either UV-A or UV-B absorbers. UV-A absorbers generally absorb radiation in the 320 to 400 nm region of the ultraviolet spectrum. UV-A absorbers include anthranilates, benzophenones, and dibenzoyl methanes. UV-B absorbers generally absorb radiation in the 280 to 320 nm region of the ultraviolet spectrum. UV-B absorbers include p-aminobenzoic acid derivatives, camphor derivatives, cinnamates, and salicylates.

Classifying the chemical absorbers generally as UV-A or UV-B absorbers is accepted within the industry. However, a more precise classification is one based upon the chemical properties of the sunscreens. There are eight major classifications of sunscreen chemical properties which are discussed at length in “Sunscreens—Development, Evaluation and Regulatory Aspects,” by N. Shaath et al., 2nd. Edition, pages 269-273, Marcel Dekker, Inc. (1997). This discussion, in its entirety, is incorporated by reference herein.

The sunscreens which may be formulated according to the present invention typically comprise chemical absorbers, but may also comprise physical blockers. Exemplary sunscreens which may be formulated into the compositions of the present invention are chemical absorbers such as p-aminobenzoic acid derivatives, anthranilates, benzophenones, camphor derivatives, cinnamic derivatives, dibenzoyl methanes, diphenylacrylate derivatives, salicylic derivatives, triazine derivatives, benzimidazole compounds, bis-benzoazolyl derivatives, methylene bis-(hydroxyphenylbenzotriazole) compounds, the sunscreen polymers and silicones, or mixtures thereof. These are variously described in U.S. Pat. Nos. 2,463,264, 4,367,390, 5,166,355 and 5,237,071 and in EP-0,863,145, EP-0,517,104, EP-0,570,838, EP-0,796,851, EP-0,775,698, EP-0,878,469, EP-0,933,376, EP-0,893,119, EP-0,669,323, GB-2,303,549, DE-1,972,184 and WO-93/04665, also expressly incorporated by reference. Also exemplary of the sunscreens which may be formulated into the compositions of this invention are physical blockers such as cerium oxides, chromium oxides, cobalt oxides, iron oxides, red petrolatum, silicone-treated titanium dioxide, titanium dioxide, zinc oxide, and/or zirconium oxide, or mixtures thereof.

A wide variety of sunscreens is described in U.S. Pat. No. 5,087,445, issued to Haffey et al. on Feb. 11, 1992; U.S. Pat. No. 5,073,372, issued to Turner et al. on Dec. 17, 1991; and Chapter VIII of Cosmetics and Science and Technology by Segarin et al., pages 189 et seq. (1957), all of which are incorporated herein by reference in their entirety.

Sunscreens which may be formulated into the compositions of the instant invention are those selected from among: aminobenzoic acid, amyldimethyl PABA, cinoxate, diethanolamine p-methoxycinnamate, digalloyl trioleate, dioxybenzone, 2-ethoxyethyl p-methoxycinnamate, ethyl 4-bis(hydroxypropyl)aminobenzoate, 2-ethylhexyl-2-cyano-3,3-diphenylacrylate, ethylhexyl p-methoxycinnamate, 2-ethylhexyl salicylate, glyceryl aminobenzoate, homomethyl salicylate, homosalate, 3-imidazol-4-ylacrylic acid and ethyl ester, methyl anthranilate, octyldimethyl PABA, 2-phenylbenzimidazole-5-sulfonic acid and salts, red petrolatum, sulisobenzone, titanium dioxide, triethanolamine salicylate, N,N,N-trimethyl-4-(2-oxoborn-3-ylidene methyl)anilinium methyl sulfate, and mixtures thereof.

Sunscreens active in the UV-A and/or UV-B range that can be fabricated into particles 120 of the present invention can also include, but are not limited to: p-aminobenzoic acid; oxyethylene (25 mol) p-aminobenzoate; 2-ethylhexyl p-dimethylaminobenzoate; ethyl N-oxypropylene p-aminobenzoate; glycerol p-aminobenzoate; 4-isopropylbenzyl salicylate; 2-ethylhexyl 4-methoxycinnamate; methyl diisopropylcinnamate; isoamyl 4-methoxycinnamate; diethanolamine 4-methoxycinnamate; 3-(4′-trimethylammunium)-benzyliden-bornan-2-one methylsulfate; 2-hydroxy-4-methoxybenzophenone; 2-hydroxy-4-methoxybenzophenone-5-sulfonate; 2,4-dihydroxybenzophenone; 2,2′,4,4′-tetrahydroxybenzophenone; 2,2′-dihydroxy-4,4′dimethoxybenzophenone; 2-hydroxy-4-n-octoxybenzophenone; 2-hydroxy-4-methoxy-4′-methoxybenzophenone; -(2-oxoborn-3-ylidene)-tolyl-4-sulfonic acid and soluble salts thereof; 3-(4′-sulfo)benzyliden-bornan-2-one and soluble salts thereof; 3-(4′methylbenzylidene)-d,1-camphor; 3-benzylidene-d,1-camphor; benzene 1,4-di(3-methylidene-10-camphosulfonic) acid and salts thereof (the product Mexoryl SX as described in U.S. Pat. No. 4,585,597 issued to Lange et al. on Apr. 29, 1986); urocanic acid; 2,4,6-tris[p-(2′-ethylhexyl-1′-oxycarbonyl)-anilino]-1,3,5-triazine; 2-[(p-(tertiobutylamido)anilino]-4,6-bis-[(p-(2′-ethylhexyl-1′-oxycarbonyl) anilino]-1,3,5-triazine; 2,4-bis{[4-(2-ethyl-hexyloxy)]-2-hydroxyl-phenyl}-6-(4-methoxy-phenyl)-1,3,5-triazine (“TINOSORB S”™ by Ciba); polymer of N-(2 et 4)-[(2-oxoborn-3-yliden)methyl]benzyl]-acrylamide; 1,4-bisbenzimidazolyl-phenylen-3,3′,5,5′-tetrasulfonic acid and salts thereof; benzalmalonate-substituted polyorganosiloxanes; benzotriazole-substituted polyorganosiloxanes (Drometrizole Trisiloxane); dispersed 2,2′-methylene-bis-[6-(2H-benzotriazol-2-yl)-4-(1,1,3,3-tetramethylbutyl)phenol] such as MIXXIM BB/100™ by Fairmount Chemical; micronized in dispersed form thereof such as TINOSORB M™ by Ciba-Geigy, solubilized 2,2′-methylene-bis-[6-(2H-benzotriazol-2-yl)-4-(methyl)phenol] such as MIXXIM BB/200™ by Fairmount Chemical; combinations thereof, and the like.

Further compositions or ingredients that can be formed into particles 120 of the present invention include, but are not limited to: dibenzoyl methane derivatives other than avobenzone as described, for example, in FR-2,326,405, FR-2,440,933 and EP-0,114,607, hereby expressly incorporated by reference; other dibenzoyl methane sunscreens other than avobenzone include (whether singly or in any combination): 2-methyldibenzoylmethane; 4-methyldibenzoylmethane; 4-isopropyldibenzoylmethane; 4-tert.-butyldibenzoylmethane; 2,4-dimethyldibenzoylmethane; 2,5-dimethyldibenzoylmethane; 4,4′-diisopropyldibenzoylmethane; 4,4′-dimethoxydibenzoylmethane; 2-methyl-5-isopropyl-4′-methoxydibenzoylmethane 2-methyl-5-tert.-butyl-4′-methoxydibenzoylmethane; 2,4-dimethyl-4′-methoxydibenzoylmethane; 2,6-dimethyl-4-tert.-butyl-4′-methoxydibenzoylmethane; combinations thereof, and the like.

Additional sunscreen compositions that can be formed into the particles of the present invention include, but are not limited to, those described in pages 2954-2955 of the International Cosmetic Ingredient Dictionary and Handbook (9th ed. 2002), which is incorporated herein by reference.

According to the present invention, compositions that can be fabricated into the particles of the present invention can further include at least one filler. As used herein, the term “filler” means any particle (e.g., a particle of the present invention) that is solid at room temperature and atmospheric pressure, used alone or in combination, which does not react chemically with the various ingredients of the emulsion and which is insoluble in these ingredients, even when these ingredients are raised to a temperature above room temperature and in particular to their softening point or their melting point. In an embodiment, the at least one filler has a melting point at least greater than 1700 degree C., for example, greater than 2000 degree C. In another embodiment, the at least one filler may have an apparent diameter ranging from about 0.01 micrometer to about 150 micrometers. In other embodiments, the filler particle can have a diameter of between from about 0.5 micrometers to about 120 micrometers or from about 1 micrometer to about 80 micrometers. An apparent diameter corresponds to the diameter of the circle into which the elementary particle 120 fits along its shortest dimension (thickness for leaflets). Further, the at least one filler may be absorbent, i.e., capable in particular of absorbing the oils of the composition and also the biological substances secreted by the skin, may be surface-treated, e.g., to make it lipophilic, and/or may be porous so as to absorb the sweat and/or sebum secreted by the skin.

According to alternative embodiments, the at least one filler may be chosen from inorganic and organic fillers, and may have any shape such as lamellar, spherical and/or oblong. Non-limiting examples of the at least one inert filler include talc, mica, silica, kaolin, polyamide powders (such as NYLON® powder, and such as the product sold by Atochem as ORGASOL®), poly-.beta.-alanine powders, polyethylene powders, acrylic polymer powders (such as polymethyl methacrylate (PMMA) powder, for instance the product sold by Wacker as COVABEAD LH-85™ (particle size 10-12 micrometer) and the acrylic acid copolymer powder sold by Dow Corning as POLYTRAP®), polytetrafluoroethylene (TEFLON® by DuPont) powders, lauroyllysine, boron nitride, silica, kaolin, starch, starch derivatives, hollow polymer microspheres (such as those hollow polymer microspheres formed from polyvinylidene chloride and acrylonitrile, for instance the product sold by Nobel Industrie as EXPANCEL®), and polymerized silicone microspheres (such as those polymerized silicone microspheres sold by Toshiba as TOSPEARL®), precipitated calcium carbonate, magnesium carbonate and hydrocarbonate, hydroxyapatite, hollow silica microspheres (such as the product sold by Maprecos as SILICA BEADS®), glass microcapsules, ceramic microcapsules, and polyester particles.

Other embodiments of the inventions may include other cosmetically or dermatologically acceptable additional ingredients in the particles 120 such as thickeners, preservatives, or biological actives and any other ingredient that a person of ordinary skill in the art may identify. These and other additional ingredients may be found in the International Cosmetic Ingredient Dictionary and Handbook (9th ed. 2002), which is incorporated herein by reference.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

In some embodiments, particles 120 according to the present invention include particles that include in whole or in part, sugars, sugar-derivatives, Block-copolymers, anti-polluting agents, hair removal compositions, artificial tanning compositions, such as iron oxide, dehydration resistance compositions, combinations thereof, and the like. Some embodiments include devices and methods for dispensing compositions of particles. Some devices can include, but are not limited to, pads, sprayers, pumps, shakers, brushes, combinations thereof, and the like. In other embodiments, particles of the present invention are dispersed in topical dispersions. In some embodiments, the topical dispersions dry on contact. In some embodiments, some topical dispersions include dispersions of particles in silicone.

In some embodiments, particles 120 of the present invention include particles containing at least one of following ingredients: Mexoryl SX—active ingredient in sunscreen since 1993, UVA filter; Aminexil—active ingredient in hairloss prevention, 1996; Nanosomes—for transport of active ingredients into the skin; Ceramide R—rebuilding hair fibers; Salicylic Acid—topical acne active ingredient—drying agent; Vitamin C—radiance renewal action on surface; Sphingo-lipid—a ceramide which improves the barrier function of skin be replenishing the skin's supply of lipids; Adenoxine—crease reducing complex, smoothes skin and improves wrinkle appearance; Fibrocyclamide—replenishes skin's elasticity, reinforces skin's support system firming up skin; Filladyn—skin hydro-captor, helps skin continuously hydrate for long periods of time; Phytovone—combination of soya proteins and Wild Yam, visibly improves the loss of skin density; Acexamic acid—relieves skin tightness; Jojoba pearls—smooth skin; Lipids; Urea—allows skin to retain water; Lactate A—allow skin to retain water; Adrenalyse—help reduce the appearance of fatty dimples; UBIQUINONE®−cleanser; micro-particles—exfolliant; Mineral salts; trace elements; Manganese; Polyfructol; Serine—natural hydro-fixer; Zincadone A; N.M.S.—natural hydrofixers; pro-vitamin B5; Retinol; Biophenone; Lissyne™; Phyto-Comples Concentrate; Escinine; Flexilip; Calendula; Antioxidants; Vitamin E; Sodium hyaluronate; Alpha hydroxyl acids; microparticle oil absorbers; Glycolic acid; Kojic acid; Mandelic acid; Anti-bacterial; Anti-fungal; Anti-inflammatory; Botanical extracts; L-ascorbic acid; Alpha tocopherol; Centella asiatica; Ecotin; Titanium dioxide; Talc; Nylon-12; Mica; Hamamelis extract; Lotus flower extract; Hydra-claryl; Calcium; Fruit acids; Vitamin B3; Vitamin B6; Fructose; Glucose; Pyrithione zinc; combinations thereof, and the like.

In some embodiments, particles 120 according to the present invention include particles fabricated for personal care products such as makeup that includes, foundation (liquid or powder), powder finish, eye-shadow, blush, eye liner, lip liner, lip stick, lip gloss, mascara, blemish concealer, tanning cream, fingernail polish, combinations thereof, and the like.

In some embodiments, particles 120 according to the present invention include particles fabricated for personal care products such as hair products such as hair coloring, toning, glossing, and balancing, temporary products, non-permanent products, permanent (multi faceted, fade resistant, ultra protective, plus highlights), highlights (high precision, high intensity), pigments, combinations thereof, and the like.

In some embodiments, particles according to the present invention include particles fabricated for personal care products such as skin products such as toners, pore tightening astringents, skin renewing toner, pore clarifying, soothing tone, energizing toner, moisturizer, wrinkle defense, reactivating, vitamin/mineral cream, sunscreen, cleansers such as foaming, anti-clogging, deep-clean, anti-fatigue cleaners, combinations thereof, and the like.

In some embodiments, particles according to the present invention include particles fabricated for personal care products such as hair care products such as shampoo, conditioner, hair spray, mousse, gel, anti-frizz, color-boosting products, combinations thereof, and the like. In some embodiments, particles according to the present invention include particles fabricated for personal care products such as cologne, perfume, shaving products, aftershave, deodorant, combinations thereof, and the like.

Further uses and applications of particles 120 fabricated according to the present invention can be found in U.S. Pat. Nos. 7,030,985; 6,958,155; 6,946,123; 6,126,929; 6,548,051; 6,432,417; 5,776,241; 5,643,672; 5,690,945; 5,637,291; 5,776,947; 5,679,326; 5,538,717; 6,946,124; 6,869,599; 6,703,028; 6,669,389; 6,667,378; 6,638,519; 6,531,113; 6,280,765; 6,258,345; 6,254,876; 6,165,446; 6,132,736; 6,083,494; 6,071,524; 5,914,117; 5,866,108; 5,814,322; 5,725,847; 5,000,937; 6,692,730; 6,548,050; 6,544,532; 7,029,662; 5,824,296; 7,011,823; 6,979,469; 6,964,773; 6,946,518; 6,565,839; 6,464,969; 6,344,205; 6,333,053; 5,223,559; 7,030,985; 7,023,552; 7,022,316; 7,008,935; 6,958,155; 6,953,484; 6,946,124; 6,906,106; 6,902,737; 6,896,889; 6,894,012; 6,869,599; 6,855,311; 6,846,479; 6,846,333; 6,824,765; 6,824,764; 6,818,206; 6,811,770; 6,793,916; 6,793,913; 6,776,980; 6,761,881; 6,749,839; 6,740,313; 6,726,916; 6,689,371; 6,682,748; 6,641,802; 6,635,239; 6,630,131; 6,627,180; 6,596,264; 6,555,096; 6,541,018; 6,531,113; 6,515,178; 6,464,990; 6,461,625; 6,436,377; 6,436,376; 6,432,389; 6,419,946; 6,419,908; 6,416,768; 6,416,748; 6,413,527; 6,409,998; 6,406,685; 6,403,704; 6,403,061; 6,379,655; 6,375,960; 6,375,936; 6,361,782; 6,359,175; 6,335,022; 6,333,026; 6,326,013; 6,319,959; 6,296,839; 6,296,835; 6,287,543; 6,274,150; 6,267,950; 6,254,877; 6,254,876; 6,251,375; 6,231,839; 6,228,377; 6,221,344; 6,207,175; 6,207,173; 6,203,802; 6,200,579; 6,183,728; 6,171,579; 6,166,093; 6,146,649; 6,130,213; 6,126,948; 6,123,960; 6,080,415; 6,066,328; 6,060,041; 6,033,648; 6,024,944; 5,993,831; 5,985,925; 5,985,250; 5,972,354; 5,961,989; 5,958,387; 5,955,091; 5,954,871; 5,948,415; 5,945,095; 5,939,079; 5,939,053; 5,932,194; 5,928,629; 5,925,364; 5,919,469; 5,910,313; 5,904,918; 5,863,522; 5,858,334; 5,851,517; 5,846,550; 5,833,967; 5,795,565; 5,788,973; 5,788,955; 5,776,497; 5,776,440; 5,776,241; 5,762,912; 5,756,110; 5,753,209; 5,733,895; 5,730,993; 5,695,747; 5,693,329; 5,690,917; 5,690,915; 5,688,527; 5,686,085; 5,684,178; 5,679,829; 5,674,504; 5,670,139; 5,660,839; 5,658,555; 5,645,609; 5,643,581; 5,643,557; 5,626,868; 5,626,853; 5,618,520; 5,616,331; 5,607,664; 5,583,234; 5,571,700; 5,556,617; 5,547,658; 5,496,543; 5,451,254; 5,449,403; 5,443,840; 5,034,419; U.S. Published application nos.: 20060057092A1; 20060045895A1; 20050276770A1; 20060039938A1; 20050238609A1; 20050238604A1; 20050118122A1; 20050106196A1; 20050031699A1; 20040152620A1; 20040146473A1; 20040137028A1; 20030072602A1; 20020176843A1; and Foreign patent no. GB2107186A, each of which is incorporated herein by reference in its entirety, including all references cited therein.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### 200 nm Trapezoidal Particles Made from Various Matrix Materials

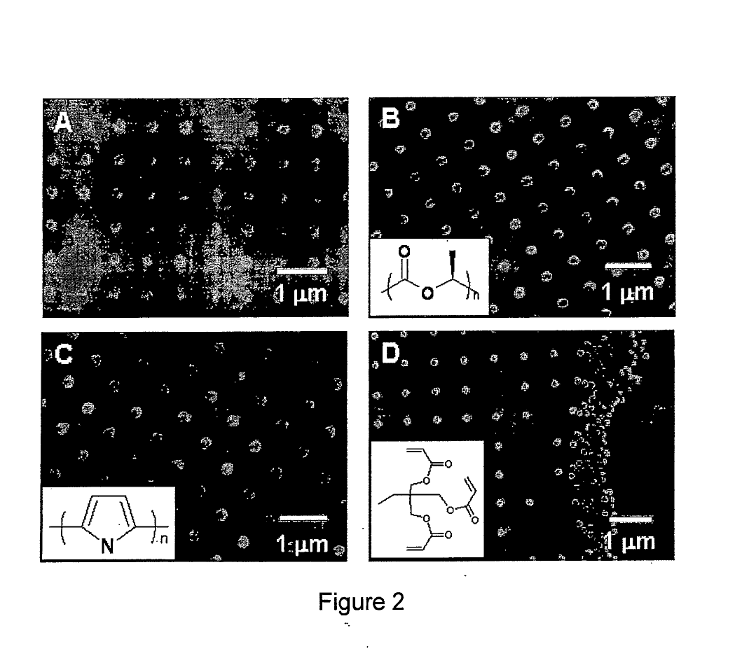

To demonstrate the utility and flexibility of the PRINT™ process, shape specific organic particles composed of three different materials were generated from a commercially available silicon template (FIG. 2A) that is composed of a 2 dimensional array of 200 nm trapezoids. Elastomeric PFPE replica molds of the silicon master templates were generated by pouring a PFPE-dimethacrylate (PFPE-DMA) containing 1-hydroxycyclohexyl phenyl ketone over the silicon substrate patterned with 200-nm trapezoidal shapes. A poly(dimethylsiloxane) mold is used to confine the liquid PFPE-DMA to the desired area. The apparatus was then subjected to UV light (λ=365 nm) for 10 minutes while under a nitrogen purge. The fully cured PFPE-DMA mold was then released from the silicon master. This process was repeated to obtain several molds of the same master.

To fabricate monodisperse PLA particles using the PRINT™ process, one gram of (3S)-cis-3,6-dimethyl-1,4-dioxane-2,5-dione (melting point 92° C.) was heated to 110° C. and approximately 20 μL of stannous octoate catalyst/initiator is added to the liquid monomer. Flat, uniform, non-wetting surfaces are generated by treating a silicon wafer cleaned with “piranha” solution (1:1 concentrated sulfuric acid: 30% hydrogen peroxide (aq) solution) with trichloro(1H, 1H, 2H, 2H-perfluorooctyl) silane via vapor deposition in a desiccator for 20 minutes. Following this, 50 μL of molten Lactic acid containing catalyst is then placed on the treated silicon wafer preheated to 110° C. and the patterned PFPE mold is placed on top of it. A small pressure is applied to the top of the mold with a planar surface to push out excess monomer. The entire apparatus is then placed in an oven at 110° C. for 15 hours. After polymerization was achieved, the PFPE mold and the flat, nonwetting substrate were separated to reveal monodisperse 200 nm trapezoidal particles (FIG. 2B).

To further demonstrate the versatility and breadth of the PRINT™ process technique, we chose to generate specifically shaped particles of 200 nm trapezoids from poly(pyrrole) (PPy). PPy has been used in a variety of applications, ranging from electronic devices and sensors to cell scaffolds. We fabricated PPy particles via one-step polymerization using the following method: flat, uniform, non-wetting surfaces are generated by treating a silicon wafer cleaned with “piranha” solution (1:1 concentrated sulfuric acid: 30% hydrogen peroxide (aq) solution) with trichloro(1H, 1H, 2H, 2H-perfluorooctyl) silane via vapor deposition in a desiccator for 20 minutes. Separately, 50 μL of a 1:1 v:v solution of tetrahydrofuran:pyrrole is added to 50 μL of 70% perchloric acid (aq). A clear, homogenous, brown solution quickly forms and develops into black, solid, polypyrrole in 15 minutes. A drop of this clear, brown solution (prior to complete polymerization) is placed onto a treated silicon wafer, the PFPE mold is placed on top, and pressure is applied with a planar surface to remove excess solution. The apparatus is then placed into a vacuum oven for 15 h to remove the THF and water. Particles are observed using scanning electron microscopy (SEM) (see FIG. 2C) after release of the vacuum and separation of the PFPE mold and the treated silicon wafer.

Trapezoidal trimethylopropane triacrylate (TMPTA) particles were also generated using a photopolymerization technique. TMPTA is blended with 1 wt % of a photoinitiator, 1-hydroxycyclohexyl phenyl ketone. Uniform, non-wetting surfaces are generated by pouring a PFPE-dimethacrylate (PFPE-DMA) containing 1-hydroxycyclohexyl phenyl ketone over a silicon wafer. The wafer was then subjected to UV light (λ=365 nm) for 10 minutes while under a nitrogen purge. The fully cured PFPE-DMA substrate was then released from the silicon master. Following this, 50 μL of TMPTA is then placed on the PFPE substrate and the patterned PFPE mold placed on top of it. The substrate is then placed on a flat surface and a small pressure is applied to push out excess TMPTA. The entire apparatus is then subjected to UV light (λ=365 nm) for ten minutes while under a nitrogen purge. Particles are observed after separation of the PFPE mold and the treated silicon wafer using scanning electron microscopy (SEM). A flat blade was pushed along the surface to gather the fabricated 200 nm particles (see FIG. 2D).

Particles of the same unique dimensions made using these three different polymerization methods were evaluated using scanning electron microscopy and atomic force microscopy. The NIH Image program was used to measure the particle dimensions on the micrographs and compare them to images of the master template.

### Example 2

### Fabrication of PEG Particles of Different Shapes

Poly(ethylene glycol) (PEG) is a material of tremendous interest to the biotechnology community due to its commercial availability, nontoxic nature, and biocompatibility. Here, the PRINT™ process was utilized to produce monodisperse, micro- and nanometer scale PEG particles in a variety of shapes by molding a PEG-diacrylate liquid monomer followed by room temperature photopolymerization. Because the morphology of the particles is controlled by the master, it is possible to generate complex particles on a variety of length scales.

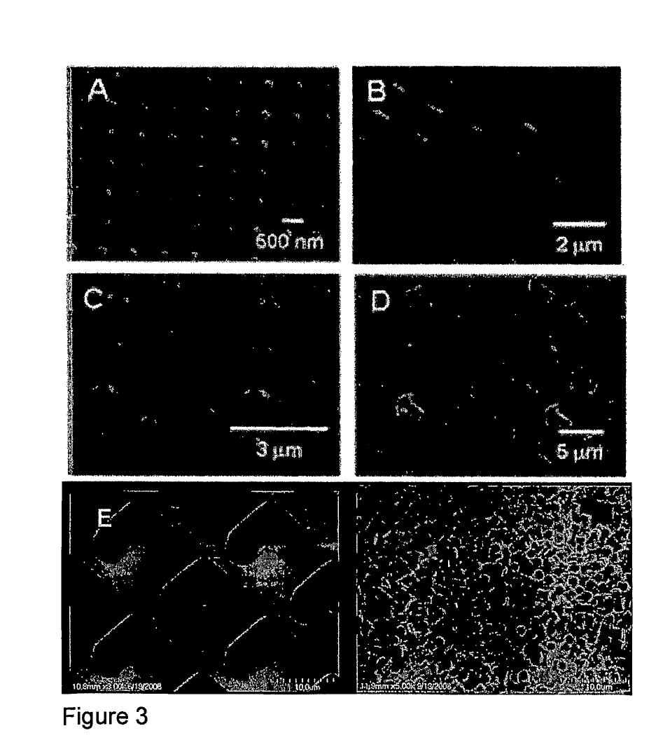

A patterned perfluoropolyether (PFPE) molds are generated by pouring a PFPE-dimethacrylate (PFPE-DMA) containing 1-hydroxycyclohexyl phenyl ketone over a silicon substrate patterned with the desired shape. The silicon masters used include: 200 nm trapezoidal features; 200 nm×800 nm bars; 500 nm conical features that are <50 nm at the tip; 3 μm arrows; 10 μm boomerangs; and 600 nm cylinders. The master coated with uncured PFPE was then subjected to UV light (λ=365 nm) for 10 minutes while under a nitrogen purge. The fully cured PFPE-DMA mold was then easily released from the silicon master by peeling. Separately, a poly(ethylene glycol) (PEG) diacrylate (n=9) is blended with 1 wt % of a photoinitiator, 1-hydroxycyclohexyl phenyl ketone. Uniform, non-wetting surfaces are generated by pouring a PFPE-dimethacrylate (PFPE-DMA) containing 1-hydroxycyclohexyl phenyl ketone over a silicon wafer. The wafer was then subjected to UV light (λ=365 nm) for 10 minutes while under a nitrogen purge. The fully cured PFPE-DMA substrate was then released from the silicon master. Following this, 50 μL of PEG diacrylate is then placed on the PFPE film and the patterned PFPE mold placed on top of it. The substrate is then placed on a flat surface and a small pressure is applied to push out excess PEG-diacrylate. The pressure used was at least about 100 N/cm². The entire apparatus was then subjected to UV light (λ=365 nm) for ten minutes while under a nitrogen purge. Particles are observed after separation of the PFPE mold and the treated silicon wafer using scanning electron microscopy (SEM) (see FIG. 3).

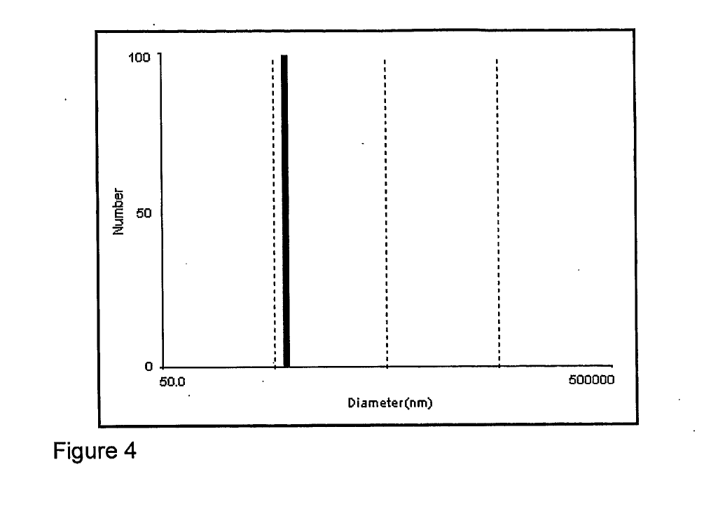

Confirmation of the structural similarity between the silicon master and replicate PEG particles was confirmed via atomic force microscopy (AFM) and scanning electron microscopy (SEM). Atomic Force Microscopy was performed on a Nanoscope IIIa/Multimode AFM in tapping mode. Dynamic light scattering (DLS) is performed on particles suspended in phosphate buffered saline solution (PBS) to look for aggregation. This technique is designed for spherical particles; however, we can use the values empirically to look for large aggregates (some non-uniformity in size will be seen at a scale smaller than that of the particle diameter due to the non-spherical shapes of the particles) An example DLS trace is given in FIG. 4, with the value measured for the particle size as 0.62±0.2 μm. The line indicates monodispersity of the particles, with no aggregation occurring.

### Example 3

### Utilizing the PRINT™ Process Technology to Create Free-Flowing Particles and Particles on a Film

The PRINT™ process technology can be used to generate a variety of products having varying forms, including free flowing particles and particles in an array on a film. The following example shows our ability to make poly(ethylene glycol) (PEG) based particles free flowing, as an array on a PEG film, and as an array on a different polymer film.

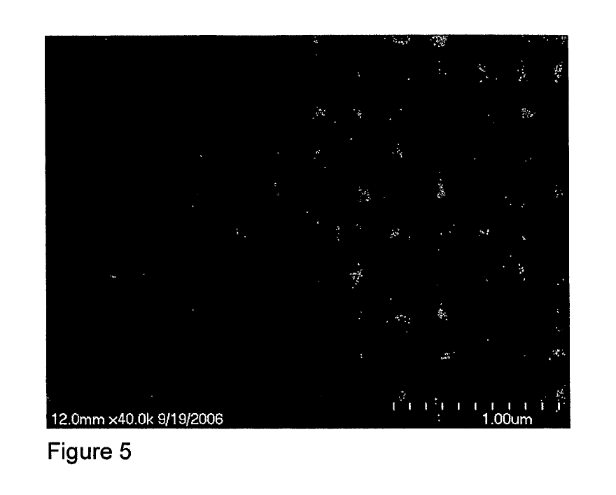

Free-flowing Particles: A patterned perfluoropolyether (PFPE) mold was generated by pouring a PFPE-dimethacrylate (PFPE-DMA) containing 1-hydroxycyclohexyl phenyl ketone over a silicon substrate patterned with 200 nm tall×200 nm diameter cylinders. The PFPE-DMA covered master was then subjected to UV light (λ=365 nm) for 3 minutes while under a nitrogen purge. The fully cured PFPE-DMA mold was then released from the silicon master. Separately, a mixture of 790 mg trimethylolpropane ethoxylate triacrylate, 200 mg polyethylene glycol carbonylimidizole monomethacrylate, and 10 mg α-α-diethoxyacetophenone was prepared. This mixture was spotted directly onto the patterned PFPE-DMA mold and covered with an unpatterned polyethylene (PE) film. The monomer mixture was pressed between the two polymer sheets, and then the PE sheet was slowly peeled from the patterned PFPE-DMA to remove any excess monomer solution from the surface of the PFPE-DMA mold. The mold was then subjected to UV light (λ=365 nm) for 2 minutes while maintaining a nitrogen purge. The particles were harvested by placing 2 mL of DMSO on the mold and scrapping the surface with a glass slide. The particle suspension was transferred to a scintillation vial. One drop of the suspension was placed on a SEM stub and the solvent was allowed to evaporate. The stub was coated with approximately 10 angstroms of gold and imaged with SEM, as shown in FIG. 5.

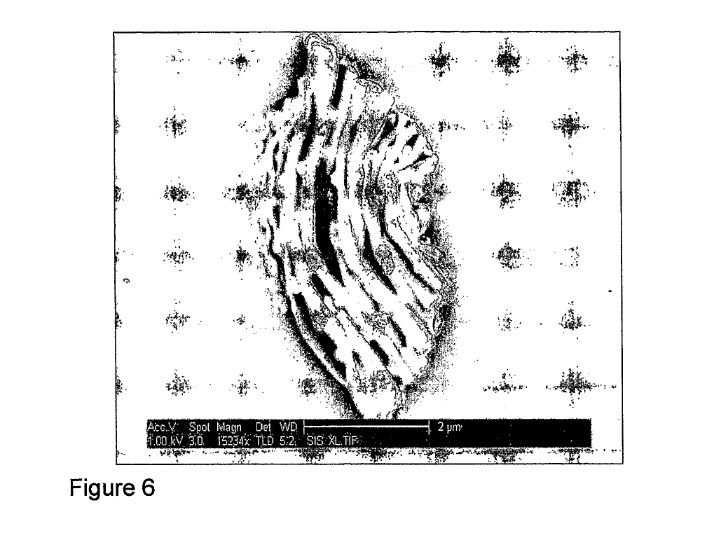

Particles on a PEG film. A patterned perfluoropolyether (PFPE) mold is generated by pouring a PFPE-dimethacrylate (PFPE-DMA) containing 1-hydroxycyclohexyl phenyl ketone over a 6 inch silicon substrate patterned with 200-nm cylindrical shapes. The substrate is then subjected to UV light (λ=365 nm) for 10 minutes while under a nitrogen purge. The fully cured PFPE-DMA mold is then released from the silicon master. Separately, a solution of 30:70 PEG monomethacrylate:PEG diacrylate is formulated with 1 wt % photoinitiator. Following this, 200 μL of this PEG solution is then placed on an untreated silicon wafer and the patterned PFPE mold placed on top of it. The substrate is then placed on a flat substrate and a small pressure is applied to push out excess PEG solution. The entire apparatus is then subjected to UV light (λ=365 nm) for ten minutes while under a nitrogen purge. PEG particles connected by a PEG film will be observed after separation of the PFPE mold and the silicon wafer using scanning electron microscopy. Dragging a blade across the surface yields a rolled up film as shown in FIG. 6.

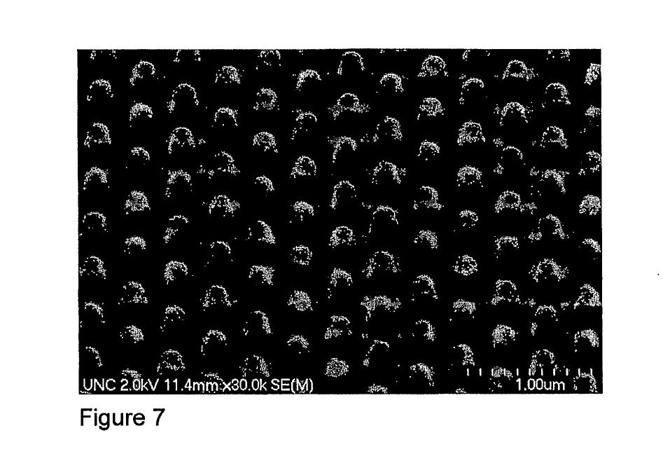

Particles on a cyanoacrylate film. A patterned perfluoropolyether (PFPE) mold is generated by pouring a PFPE-dimethacrylate (PFPE-DMA) containing 2,2-diethoxyacetophenone over a silicon substrate patterned with 200 nm cylindrical shapes. The apparatus is then subjected to a nitrogen purge for 10 minutes before the application of UV light (λ=365 nm) for 10 minutes while under a nitrogen purge. The fully cured PFPE-DMA mold is then released from the silicon master. Separately, a poly(ethylene glycol) (PEG) diacrylate (n=9) is blended with 28 wt % PEG methacrylate (n=9), 2 wt % azobisisobutyronitrile (AIBN), and 0.25 wt % rhodamine methacrylate. Flat, uniform, non-wetting surfaces are generated by coating a glass slide with PFPE-dimethacrylate (PFPE-DMA) containing 2,2-diethoxyacetophenone. The slide is then subjected to a nitrogen purge for 10 minutes, then UV light is applied (λ=365 nm) while under a nitrogen purge. The flat, fully cured PFPE-DMA substrate is released from the slide. Following this, 0.1 mL of the monomer blend is evenly spotted onto the flat PFPE-DMA surface and then the patterned PFPE-DMA mold placed on top of it. The surface and mold are then placed in a molding apparatus and a small amount of pressure is applied to remove any excess monomer solution. The entire apparatus is purged with nitrogen for 10 minutes, then subjected to UV light (λ=365 nm) for 10 minutes while under a nitrogen purge. Neutral PEG nanoparticles are observed after separation of the PFPE-DMA mold and substrate using scanning electron microscopy (SEM). A thin layer of cyanoacrylate monomer is sprayed onto the PFPE-DMA mold filled with particles. The PFPE-DMA mold is immediately placed onto a glass slide and the cyanoacrylate is allowed to polymerize in an anionic fashion for one minute. The mold is removed and the particles are embedded in the adhesive layer, as shown in FIG. 7.

### Example 4

### Identification of PRINT™ Process Particles Using Nano-Scale “Defects”

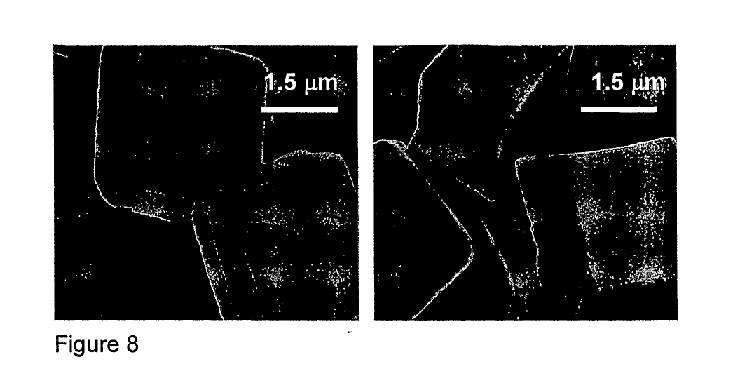

The PRINT™ process inherently introduces structural features from the silicon masters that are transferred to the mold and subsequently to the particles during PRINT™ process fabrication. Here, a Bosch-type etch is used to process a master which introduces a recognizable pattern (“Bosch etch lines”) on the sidewalls of individual particles. Bosch etching is one of many techniques used to fabricate wafers, most of which leave residual “defects” on the sidewalls of the features or surface. FIG. 8 shows distinct particles derived from the masters that show a similar sidewall pattern resulting from the specific Bosch-type etch process used on the master. In this case, this pattern can be recognized using SEM imaging and identifies these particles as originating from the same master.

A patterned perfluoropolyether (PFPE) mold is generated by pouring a PFPE-dimethacrylate (PFPE-DMA) containing 1-hydroxycyclohexyl phenyl ketone over a silicon substrate patterned with 3-elm cubical shapes at a 1 μm depth. The substrate is then subjected to a nitrogen purge for 10 minutes, then UV light (λ=365 nm) is applied for 10 minutes while under a nitrogen purge. The fully cured PFPE-DMA mold is then released from the silicon master. A PFPE-DMA mold is made from a master patterned with 2 μm deep cubical shapes. Separately, TMPTA is blended with 1 wt % of a photoinitiator, 1-hydroxycyclohexyl phenyl ketone. Flat, uniform, non-wetting surfaces are generated by coating a glass slide with PFPE-DMA containing 1-hydroxycyclohexyl phenyl ketone. The slide is then subjected to a nitrogen purge for 10 minutes, then UV light (λ=365 nm) is applied for 10 minutes while under a nitrogen purge. The flat, fully cured PFPE-DMA substrate is released from the slide. Following this, 0.1 mL of TMPTA is then placed on the flat PFPE-DMA substrate and the patterned PFPE mold placed on top of it. The substrate is then placed in a molding apparatus and a small pressure is applied to push out excess TMPTA. The entire apparatus is then purged with nitrogen for 10 minutes, then subjected to UV light (λ=365 nm) for 10 minutes while under a nitrogen purge. TMPTA particles are observed after separation of the PFPE-DMA mold and substrate using optical microscopy. A drop of n-vinyl-2-pyrrolidone containing 5% photoinitiator, 1-hydroxycyclohexyl phenyl ketone, is placed on a clean glass slide. The PFPE-DMA mold containing particles is placed patterned side down on the n-vinyl-2-pyrrolidone drop. The slide is subjected to a nitrogen purge for 5 minutes, then UV light (λ=365 nm) is applied for 5 minutes while under a nitrogen purge. The slide is removed, and the mold is peeled away from the polyvinyl pyrrolidone and particles. Particles on the polyvinyl pyrrolidone were observed with optical microscopy. The polyvinyl pyrrolidone film containing particles was dissolved in water. Dialysis was used to remove the polyvinyl pyrrolidone, leaving an aqueous solution containing TMPTA particles. Samples dispersions from the 1 μm and 2 μm deep master are dropped on an SEM stub and the water allowed to evaporate in a vacuum oven. The particles were coated with ˜10 Å gold-palladium and imaged with SEM.

### Example 5

### Fabrication of 2×2×1 μm Fluorescently Tagged Positively Charged PEG-Based Particles

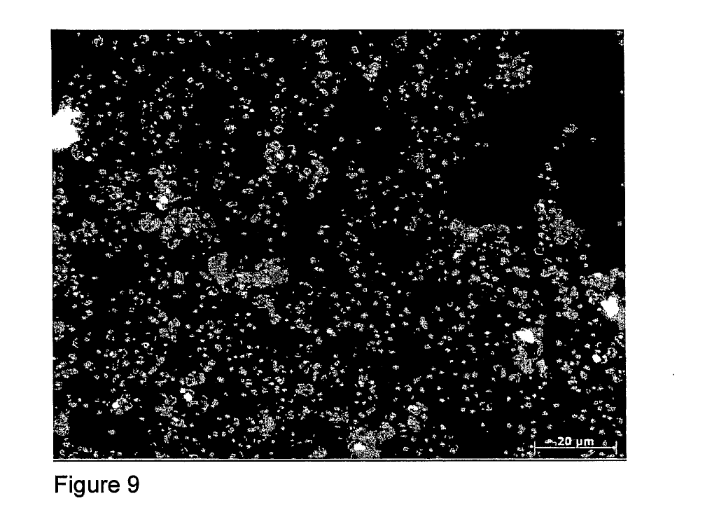

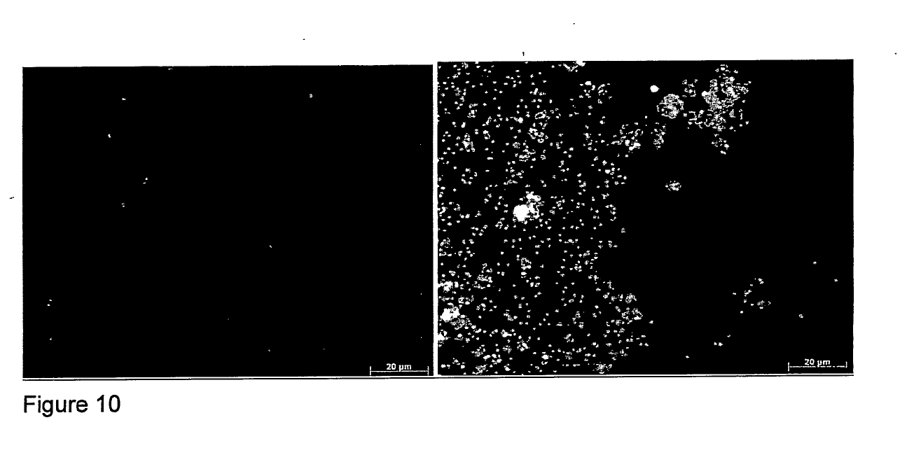

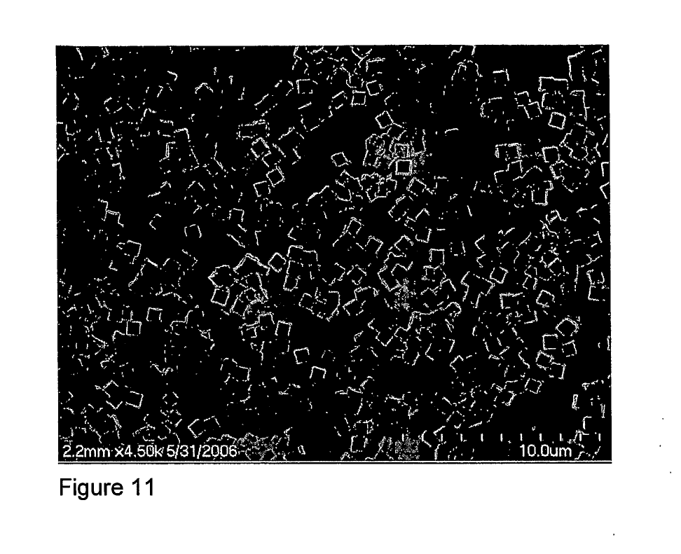

A silicon substrate patterned with 2×2×1 μm rectangular shapes is encased in an airtight UV-transparent mold maker. A patterned perfluoropolyether (PFPE) mold is generated by adding 10 mL of PFPE-dimethacrylate (PFPE-DMA) containing 2, 2-diethoxyacetophenone into the mold maker in between the patterned silicon substrate and the UV transparent lid. As the PFPE-DMA solution is added, air is pushed out leaving only the PFPE-DMA solution. The apparatus is then subjected to UV light (λ=365 nm) for 15 minutes. The fully cured PFPE-DMA mold is then released from the silicon master in the mold maker. Similarly, a flat, uniform, non-wetting surface is generated by encasing a blank silicon wafer into the airtight UV-transparent surface maker. The non-patterned perfluoropolyether (PFPE) surface is generated by adding 10 mL of PFPE-dimethacrylate (PFPE-DMA) containing 2,2-diethoxyacetophenone into the surface maker in between the non-patterned silicon substrate and the UV transparent lid. As the PFPE-DMA solution is added, air is pushed out leaving only the PFPE-DMA solution. The apparatus is then subjected to UV light (λ=365 nm) for 15 minutes. The fully cured PFPE-DMA surface is then released from the silicon surface in the surface maker. Separately, a poly(ethylene glycol) (PEG) diacrylate (n=9) (90%) is blended with amino ethyl trimethylammonium chloride (AETMAC) (10%). To this solution was added ethanol, water, hydroxyl cyclohexyl phenyl ketone initiator, and an oligonucleotide with the sequence GCT ATT ACC TTA ACC CAG containing a 3′ fluorescein label. Final solution composition was: 3.90% AETMAC, 33.21% PEG-diacrylate, 1.90% initiator, 0.04% oligo cargo, 38.93% H₂O, and 22.02% EtOH. Following this, 0.1 mL of the above monomer blend is evenly spotted onto the flat PFPE-DMA surface and then the patterned PFPE-DMA mold placed on top of it. Pressure is applied with a roller for a few strokes to help spread the monomer solution. The surface and mold are then placed atop a PDMS dome under a UV light with an attached pressure clamp (particle maker). Once inside the particle maker, the apparatus is purged with nitrogen for 6 minutes at 50 kPa. A pressure of 1 ton is applied to the mold and surface to remove any excess monomer solution. At this point, nitrogen flow is shut off. After 1 hour of pressing, the entire apparatus is subjected to UV light (λ=365 nm) for 45 minutes. After curing, the mold and surface are separated to reveal discrete 2×2×1 micrometer oligonucleotide containing particles in the mold observable by light microscopy. The harvesting process begins by dispersing a thin layer of cyanoacrylate monomer onto the PFPE-DMA mold filled with particles. The PFPE-DMA mold is immediately placed onto a glass slide and the cyanoacrylate is allowed to polymerize in an anionic fashion for one minute. The mold is removed and the particles are embedded in the soluble adhesive layer, which provides isolated, harvested colloidal particle dispersions upon dissolution of the soluble adhesive polymer layer in acetone. Particles embedded in the harvesting layer, or dispersed in acetone can be visualized by light microscopy or SEM. The fluorescently labeled oligonucleotide cargo can be visualized using a fluorescent lamp attached to the light microscope. The dissolved poly(cyanoacrylate) can remain with the particles in solution, or can be removed via centrifugation. As shown in FIG. 9, the harvested 2×2×1 μm positively charged particles contain the fluorescent oligonucleotide condensed inside. FIG. 10 shows the same region imaged by both DIC and fluorescent light microscopy. FIG. 11 contains SEM images of oligonucleotides in positively charged particles.

### Example 6

### Fabrication of 2×2×1 μm Fluorescently Tagged Neutral Peg-Based Particles

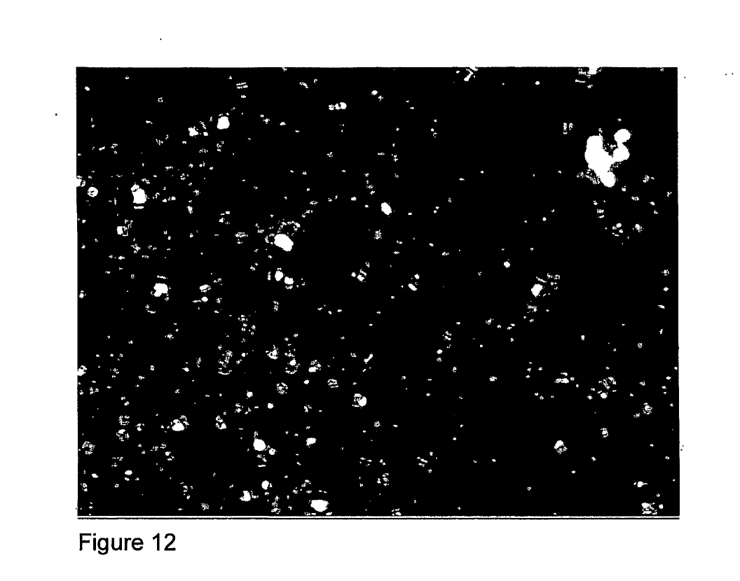

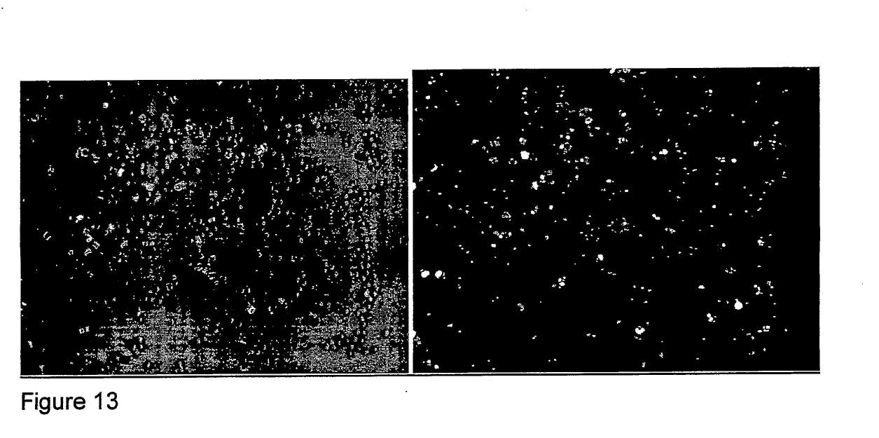

A silicon substrate patterned with 2×2×1 μm rectangular shapes is encased in an airtight UV-transparent mold maker. A patterned perfluoropolyether (PFPE) mold is generated by adding 10 mL of PFPE-dimethacrylate (PFPE-DMA) containing 2, 2-diethoxyacetophenone into the mold maker in between the patterned silicon substrate and the UV transparent lid. As the PFPE-DMA solution is added, air is pushed out leaving only the PFPE-DMA solution. The apparatus is then subjected to UV light (λ=365 nm) for 15 minutes. The fully cured PFPE-DMA mold is then released from the silicon master in the mold maker. Similarly, a flat, uniform, non-wetting surface is generated by encasing a blank silicon wafer into the airtight UV-transparent surface maker. The non-patterned perfluoropolyether (PFPE) surface is generated by adding 10 mL of PFPE-dimethacrylate (PFPE-DMA) containing 2,2-diethoxyacetophenone into the surface maker in between the non-patterned silicon substrate and the UV transparent lid. As the PFPE-DMA solution is added, air is pushed out leaving only the PFPE-DMA solution. The apparatus is then subjected to UV light (λ=365 nm) for 15 minutes. The fully cured PFPE-DMA surface is then released from the silicon surface in the surface maker. Separately, a poly(ethylene glycol) (PEG) diacrylate (n=9) is blended with ethanol, water, hydroxyl cyclohexyl phenyl ketone initiator, and an oligonucleotide with the sequence GCT ATT ACC TTA ACC CAG containing a 3′ fluorescein label. Final solution composition was: 40.53% PEG-diacrylate, 4.05% initiator, 0.03% oligo cargo, 38.34% H₂O, and 17.05% EtOH. Following this, 0.1 mL of the above monomer blend is evenly spotted onto the flat PFPE-DMA surface and then the patterned PFPE-DMA mold placed on top of it. Pressure is applied with a roller for a few strokes to help spread the monomer solution. The surface and mold are then placed atop a PDMS dome under a UV light with an attached pressure clamp particle maker). Once inside the particle maker, the apparatus is purged with nitrogen for 6 minutes at 50 kPa. A pressure of 1 ton is applied to the mold and surface to remove any excess monomer solution. At this point, nitrogen flow is shut off. After 1 hour of pressing, the entire apparatus is subjected to UV light (λ=365 nm) for 45 minutes. After curing, the mold and surface are separated to reveal discrete 2×2×1 μm oligonucleotide containing particles in the mold observable by light microscopy. The harvesting process begins by dispersing a thin layer of cyanoacrylate monomer onto the PFPE-DMA mold filled with particles. The PFPE-DMA mold is immediately placed onto a glass slide and the cyanoacrylate is allowed to polymerize in an anionic fashion for one minute. The mold is removed and the particles are embedded in the soluble adhesive layer, which provides isolated, harvested colloidal particle dispersions upon dissolution of the soluble adhesive polymer layer in acetone. Particles embedded in the harvesting layer, or dispersed in acetone can be visualized by light microscopy or SEM. The fluorescently labeled oligonucleotide cargo can be visualized using a fluorescent lamp attached to the light microscope. The dissolved poly(cyanoacrylate) can remain with the particles in solution, or can be removed via centrifugation. As shown in FIG. 12, the harvested 2×2×1 μm neutral particles contain the fluorescent oligonucleotide inside. FIG. 13 shows the same region of the harvested 2×2×1 μm neutral particles imaged by both DIC and fluorescent light microscopy.

### Example 7

### Encapsulation of Magnetite Nanoparticles Inside 500-nm Conical PEG Particles

A patterned perfluoropolyether (PFPE) mold is generated by pouring a PFPE-dimethacrylate (PFPE-DMA) containing 1-hydroxycyclohexyl phenyl ketone over a silicon substrate patterned with 500-nm conical shape. A poly(dimethylsiloxane) mold is used to confine the liquid PFPE-DMA to the desired area. The apparatus is then subjected to UV light (λ=365 nm) for 10 minutes while under a nitrogen purge. The fully cured PFPE-DMA mold is then released from the silicon master. Flat, uniform, non-wetting surfaces are generated by treating a silicon wafer cleaned with “piranha” solution (1:1 concentrated sulfuric acid: 30% hydrogen peroxide (aq) solution) with trichloro(1H, 1H, 2H, 2H-perfluorooctyl) silane via vapor deposition in a desiccator for 20 minutes. Separately, citrate capped magnetite nanoparticles were synthesized by reaction of ferric chloride (40 mL of a 1 M aqueous solution) and ferrous chloride (10 mL of a 2 M aqueous hydrochloric acid solution) which is added to ammonia (500 mL of a 0.7 M aqueous solution). The resulting precipitate is collected by centrifugation and then stirred in 2 M perchloric acid. The final solids are collected by centrifugation. 0.290 g of these perchlorate-stabilized nanoparticles are suspended in 50 mL of water and heated to 90° C. while stirring. Next, 0.106 g of sodium citrate is added. The solution is stirred at 90° C. for 30 min to yield an aqueous solution of citrate-stabilized iron oxide nanoparticles. 50 μL of this solution is added to 50 μL of a PEG diacrylate solution in a microtube. This microtube is vortexed for ten seconds. Following this, 50 μL of this PEG diacrylate/particle solution is then placed on the treated silicon wafer and the patterned PFPE mold placed on top of it. The substrate is then placed in a molding apparatus and a small pressure is applied to push out excess PEG-diacrylate/particle solution. The entire apparatus is then subjected to UV light (λ=365 nm) for ten minutes while under a nitrogen purge. Nanoparticle-containing PEG-diacrylate particles are observed after separation of the PFPE mold and the treated silicon wafer using optical microscopy.

### Example 8

### Fabrication of 200-nm Titania Particles

A patterned perfluoropolyether (PFPE) mold can be generated by pouring a PFPE-dimethacrylate (PFPE-DMA) containing 1-hydroxycyclohexyl phenyl ketone over a silicon substrate patterned with 200-nm trapezoidal shapes. A poly(dimethylsiloxane) mold can be used to confine the liquid PFPE-DMA to the desired area. The apparatus can then be subjected to UV light (λ=365 nm) for 10 minutes while under a nitrogen purge. The fully cured PFPE-DMA mold is then released from the silicon master. Separately, 1 g of Pluronic P123 is dissolved in 12 g of absolute ethanol. This solution was added to a solution of 2.7 mL of concentrated hydrochloric acid and 3.88 mL titanium (IV) ethoxide. Flat, uniform, non-wetting surfaces can be generated by treating a silicon wafer cleaned with “piranha” solution (1:1 concentrated sulfuric acid: 30% hydrogen peroxide (aq) solution) with trichloro(1H, 1H, 2H, 2H-perfluorooctyl) silane via vapor deposition in a desiccator for 20 minutes. Following this, 50 μL of the sol-gel solution can then be placed on the treated silicon wafer and the patterned PFPE mold placed on top of it. The substrate is then placed in a molding apparatus and a small pressure is applied to push out excess sol-gel precursor. The entire apparatus is then set aside until the sol-gel precursor has solidified. After solidification of the sol-gel precursor, the silicon wafer can be removed from the patterned PFPE and particles will be present.

### Example 9

### Fabrication of 200 nm Phosphatidylcholine Particles

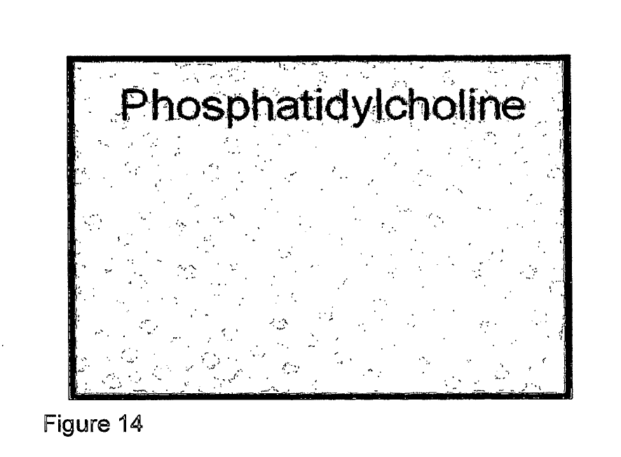

A patterned perfluoropolyether (PFPE) mold is generated by pouring a PFPE-dimethacrylate (PFPE-DMA) containing 1-hydroxycyclohexyl phenyl ketone over a silicon substrate patterned with 200-nm trapezoidal shapes. A poly(dimethylsiloxane) mold is used to confine the liquid PFPE-DMA to the desired area. The apparatus is then subjected to a nitrogen purge for 10 minutes followed by UV light (λ=365 nm) for 10 minutes while under a nitrogen purge. The fully cured PFPE-DMA mold is then released from the silicon master. Separately, flat, uniform, non-wetting surfaces are generated by treating a silicon wafer cleaned with “piranha” solution (1:1 concentrated sulfuric acid: 30% hydrogen peroxide (aq) solution) with trichloro(1H, 1H, 2H, 2H-perfluorooctyl) silane via vapor deposition in a desiccator for 20 minutes. Following this, 20 mg of the phosphatidylcholine was placed on the treated silicon wafer and heated to 60 degrees C. The substrate is then placed in a molding apparatus and a small pressure is applied to push out excess phosphatidylcholine. The entire apparatus is then set aside until the phosphatidylcholine has solidified. Particles are observed after separation of the PFPE mold and the treated silicon wafer using scanning electron microscopy (SEM) and optical microscopy, as shown in FIG. 14.

### Example 10

### Encapsulation of Avidin (66 kDa) in 160 nm Peg Particles

A patterned perfluoropolyether (PFPE) mold was generated by pouring a PFPE-dimethacrylate (PFPE-DMA) containing 1-hydroxycyclohexyl phenyl ketone over a silicon substrate patterned with 160-nm cylindrical shapes. A poly(dimethylsiloxane) mold was used to confine the liquid PFPE-DMA to the desired area. The apparatus was then subjected to UV light (λ=365 nm) for 10 minutes while under a nitrogen purge. The fully cured PFPE-DMA mold was then released from the silicon master. Flat, uniform, non-wetting surfaces are generated by treating a silicon wafer cleaned with “piranha” solution (1:1 concentrated sulfuric acid: 30% hydrogen peroxide (aq) solution) with trichloro(1H, 1H, 2H, 2H-perfluorooctyl) silane via vapor deposition in a desiccator for 20 minutes. Separately, a solution of 1 wt % avidin in 30:70 PEG monomethacrylate:PEG diacrylate was formulated with 1 wt % photoinitiator. Following this, 50 μL of this PEG/avidin solution was then placed on the treated silicon wafer and the patterned PFPE mold placed on top of it. The substrate was then placed in a molding apparatus and a small pressure is applied to push out excess PEG-diacrylate/avidin solution. The small pressure in this example was at least about 100 N/cm². The entire apparatus was then subjected to UV light (λ=365 nm) for ten minutes while under a nitrogen purge. Avidin-containing PEG particles were observed after separation of the PFPE mold and the treated silicon wafer using fluorescent microscopy.

### Example 11

### Molding of a Polystyrene Solution

A patterned perfluoropolyether (PFPE) mold is generated by pouring a PFPE-dimethacrylate (PFPE-DMA) containing 1-hydroxycyclohexyl phenyl ketone over a silicon substrate patterned with 140-nm lines separated by 70 nm. A poly(dimethylsiloxane) mold is used to confine the liquid PFPE-DMA to the desired area. The apparatus is then subjected to UV light (λ=365 nm) for 10 minutes while under a nitrogen purge. The fully cured PFPE-DMA mold is then released from the silicon master. Separately, polystyrene is dissolved in 1 to 99 wt % of toluene. Flat, uniform, surfaces are generated by treating a silicon wafer cleaned with “piranha” solution (1:1 concentrated sulfuric acid:30% hydrogen peroxide (aq) solution) and treating the wafer with an adhesion promoter. Following this, 50 μL of polystyrene solution is then placed on the treated silicon wafer and the patterned PFPE mold is placed on top of it. The substrate is then placed in a molding apparatus and a small pressure is applied to ensure a conformal contact. The entire apparatus is then subjected to vacuum for a period of time to remove the solvent. Features are observed after separation of the PFPE mold and the treated silicon wafer using atomic force microscopy (AFM) and scanning electron microscopy (SEM).

### Example 12

### Forming a Particle Containing CDI Linker

A patterned perfluoropolyether (PFPE) mold is generated by pouring a PFPE-dimethacrylate (PFPE-DMA) containing 2,2′-diethoxy-acetophenone over a silicon substrate patterned with 200 nm shapes. The apparatus is then subjected to UV light (λ=365 nm) for 15 minutes while under a nitrogen purge. The fully cured PFPE-DMA mold is then released from the silicon master. Separately, a poly(ethylene glycol) (PEG) diacrylate (n=9) is blended with 1 wt % of a photoinitiator, 2,2′-diethoxy-acetophenone. 70 μL of PEG diacrylate monomer and 30 uL of CDI-PEG monomer were mixed. Specifically, the CDI-PEG monomer was synthesized by 1,1, carbonyl imidazole was added to a solution of PEG (n=400) monomethylacrylate in chloroform. This solution was allowed to stir overnight. This solution was then further purified by an extraction with cold water. The resulting CDI-PEG monomethacrylate was then isolated via vacuum. Flat, uniform, non-wetting surfaces are generated by pouring a PFPE-dimethacrylate (PFPE-DMA) containing 2,2′-diethoxy-acetophenone over a silicon wafer and then subjected to UV light (λ=365 nm) for 15 minutes while under a nitrogen purge. Following this, 50 μL of the PEG diacrylate solution is then placed on the non wetting surface and the patterned PFPE mold placed on top of it. The substrate is then placed in a molding apparatus and a small pressure is applied to push out excess PEG-diacrylate solution. The entire apparatus is then subjected to UV light (λ=365 nm) for 15 minutes while under a nitrogen purge. Particles are observed after separation of the PFPE mold. The particles were harvested utilizing a sacrificial adhesive layer and verified via DIC microscopy. This linker can be utilized to attach an amine containing target onto the particle.

### Example 13

### Tethering Avidin to the CDI Linker

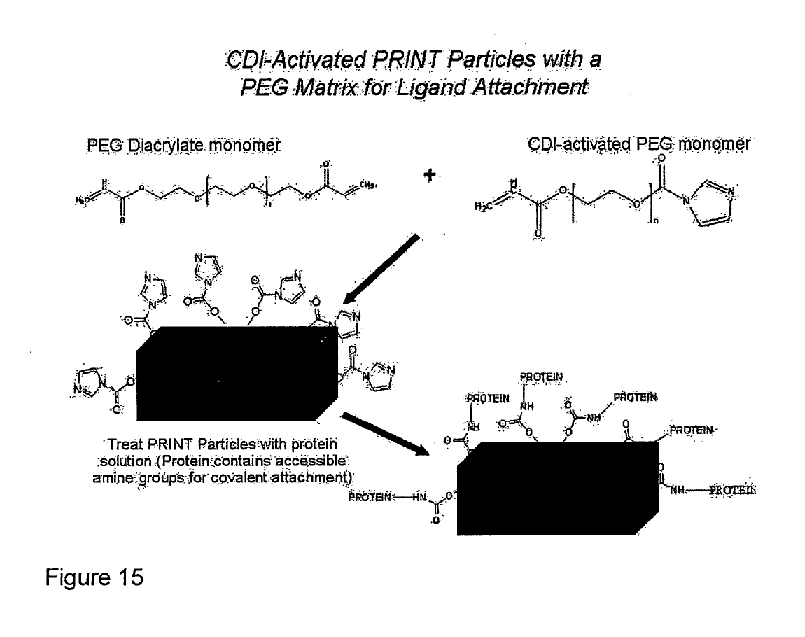

A patterned perfluoropolyether (PFPE) mold is generated by pouring a PFPE-dimethacrylate (PFPE-DMA) containing 2,2′-diethoxy-acetophenone over a silicon substrate patterned with 200 nm shapes. The apparatus is then subjected to UV light (λ=365 nm) for 15 minutes while under a nitrogen purge. The fully cured PFPE-DMA mold is then released from the silicon master. Separately, a poly(ethylene glycol) (PEG) diacrylate (n=9) is blended with 1 wt % of a photoinitiator, 2,2′-diethoxy-acetophenone. 70 μL of PEG diacrylate monomer and 30 uL of CDI-PEG monomer were mixed. Specifically, the CDI-PEG monomer was synthesized by 1,1, carbonyl imidazole was added to a solution of PEG (n=400) monomethylacrylate in chloroform. This solution was allowed to stir overnight. This solution was then further purified by an extraction with cold water. The resulting CDI-PEG monomethacrylate was then isolated via vacuum. Flat, uniform, non-wetting surfaces are generated by pouring a PFPE-dimethacrylate (PFPE-DMA) containing 2,2′-diethoxy-acetophenone over a silicon wafer and then subjected to UV light (λ=365 nm) for 15 minutes while under a nitrogen purge. Following this, 50 μL of the PEG diacrylate solution is then placed on the non wetting surface and the patterned PFPE mold placed on top of it. The substrate is then placed in a molding apparatus and a small pressure is applied to push out excess PEG-diacrylate solution. The entire apparatus is then subjected to UV light (x=365 nm) for 15 minutes while under a nitrogen purge. Particles are observed after separation of the PFPE mold. The particles were harvested utilizing a sacrificial adhesive layer and verified via DIC microscopy. These particles containing the CDI linker group were subsequently treated with and aqueous solution of fluorescently tagged avidin. These particles were allowed to stir at room temperature for four hours. These particles were then isolated via centrifugation and rinsed with deionized water. Attachment was confirmed via confocal microscopy. A schematic is given in FIG. 15.

### Example 14

### The Sol Precursor of TiO2 was Prepared by the Following Procedure

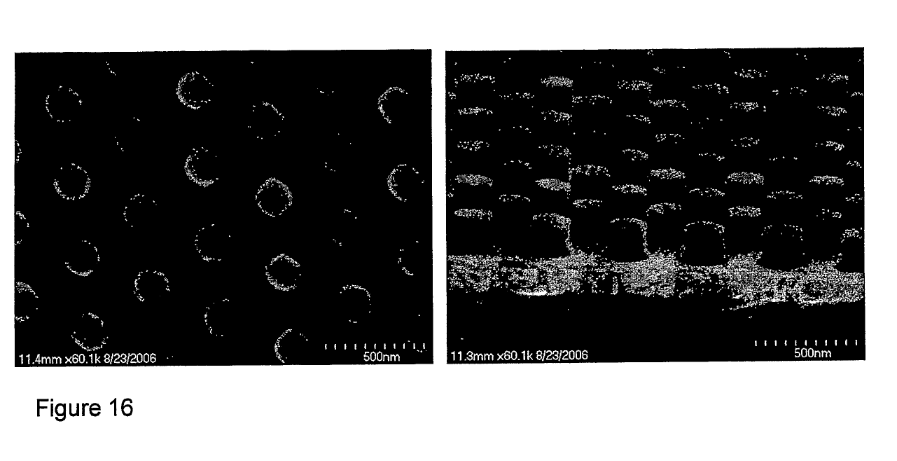

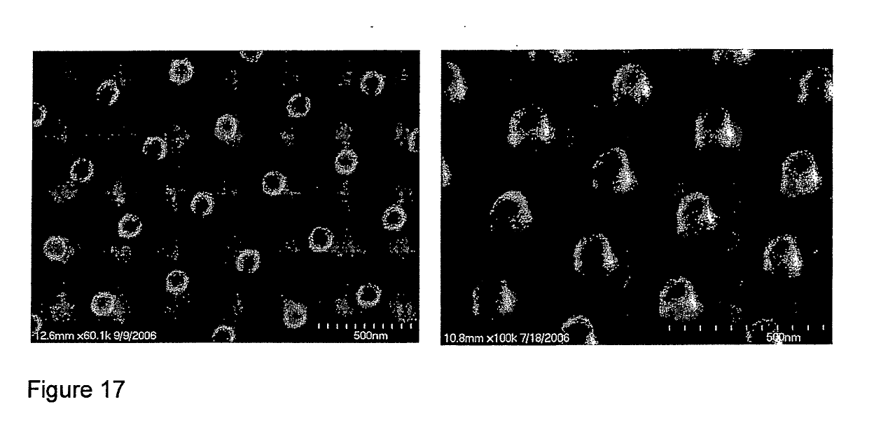

A round bottom (RB) flask equipped with a stir bar was dried at 110° C. oven before use. The RB was capped with a rubber septum and purged with nitrogen. Titanium n-butoxide (5 mL) was added to the RB under nitrogen flow. Acetylacetone (3.5 mL) was added dropwise to the reaction flask, followed by the addition of isopropanol (4 mL). Acetatic acid (0.12 mL) was added dropwise under nitrogen atmosphere to form a clean yellow mixture. The sol precursor was stirred at room temperature for 3 hr before use. To make patterned TiO2, an aliquot of the sol precursor was added onto a ITO or FTO coated substrate. A piece of FLUOROCUR™ mold with 200 nm by 200 nm features was put on top of the sol solution. The apparatus was put in a vice under pressure and kept at 110° C. oven for 3 hr. After cooling down, the TiO2 precursor had been converted to a xerogel and the FLUOROCUR™ mold was removed from the substrate. FIG. 16 shows the SEM image of patterned TiO2 xerogel prepared by this process. To convert TiO2 to the anatase form, the ITO/FTO substrate with patterned TiO2 xerogel was heated to 450° C. at a heating rate of 4° C./min and kept at 450° C. for 1 hr. The crystalline form of the calcinated TiO2 was confirmed by XRD. FIG. 17 show the SEM image of the patterned TiO2 in the anatase form after calcination.

It will be understood that various details of the presently disclosed subject matter can be changed without departing from the scope of the presently disclosed subject matter. Furthermore, the foregoing description is for the purpose of illustration only, and not for the purpose of limitation.

1. A cosmetic composition, comprising:
a dispersion of particles in a cosmetically acceptable medium wherein substantially every particle of the dispersion is:
configured and dimensioned into a predetermined three dimensional geometric shape; and
has a broadest cross-sectional dimension of less than about 100 micrometers.

2. The cosmetic composition of claim 1, wherein the particles comprise a composition of cosmetic ingredients.

3.-22. (canceled)

23. A cosmetic composition, comprising:
a cosmetic film comprising a film layer and a plurality of structures associated with the film layer wherein substantially every structure of the plurality of structures is:
configured and dimensioned into a predetermined three dimensional geometric shape; and
has a broadest cross-sectional dimension of less than about 100 micrometers.

24. The cosmetic composition of claim 23, wherein the film layer and the structures comprise the same cosmetic ingredients.

25. The cosmetic composition of claim 23, wherein a composition of the film layer is different from a composition of the structures.

26. A method for forming cosmetic particles, comprising:
providing a replica mold defining cavities having substantially uniform three dimensional geometric shapes;
introducing a cosmetic substance into the cavities of the replica mold;
hardening the substance in the cavities of the replica mold such that a particle of the cosmetic substance is formed in the cavity; and
removing the particle from the cavity of the replica mold to provide a collection of substantially uniform three-dimensional geometric shaped cosmetic particles.

27. The method of claim 26, wherein the replica mold comprises a low surface-energy polymeric material.

28. The method of claim 26, wherein the replica mold comprises a fluoropolymer.

29. The method of claim 26, wherein cavities are less than about 500 micrometers in a broadest dimension.

30.-32. (canceled)

33. The method of claim 26, wherein cavities are less than about 100 micrometers in a broadest dimension.

34. (canceled)

35. The method of claim 26, wherein cavities are less than about 10 micrometers in a broadest dimension.

36. The method of claim 26, wherein cavities are less than about 1 micrometer in a broadest dimension.

37. (canceled)

38. The method of claim 26, wherein cavities are less than about 500 nm in a broadest dimension.

39. The method of claim 26, wherein cavities are less than about 250 nm in a broadest dimension.

40. (canceled)

41. (canceled)

42. The method of claim 26, wherein cavities are less than about 100 nm in a broadest dimension.

43. (canceled)

44. (canceled)

45. The cosmetic composition of claim 1, wherein the predetermined three dimensional geometric shape is substantially cubical.

46. The cosmetic composition of claim 1, wherein the predetermined three dimensional geometric shape is substantially columnar.

47. The cosmetic composition of claim 1, wherein the predetermined three dimensional geometric shape is substantially cylindrical.

48. The cosmetic composition of claim 1, wherein the predetermined three dimensional geometric shape is substantially conical.

49. The cosmetic composition of claim 1, wherein the predetermined three dimensional geometric shape is substantially spherical.

50. The cosmetic composition of claim 1, wherein the particles are substantially equivalent in three-dimensional geometric shape.

51. The cosmetic composition of claim 1, wherein the dispersion comprises particles configured and dimensioned into a plurality of predetermined geometric shapes.

52. The cosmetic composition of claim 1, wherein the broadest cross-sectional dimension of substantially every particle of the dispersion of particles is less than about 10 micrometers.

53. The cosmetic composition of claim 1, wherein the broadest cross-sectional dimension of substantially every particle of the dispersion of particles is less than about 1 micrometer.

54. The cosmetic composition of claim 1, wherein the broadest cross-sectional dimension of substantially every particle of the dispersion of particles is less than about 250 nm.
